SEQUENCE LISTING (12 sequences)

SEQ ID NO: 1 - protein, 560 aa, Artificial
  Zika EDI MBP
MKIKTGARILALSALTTMMFSASALAKSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAIRCIGVSNRDFVEGMSGGTWVDVVLEHGGCVTVMAQDKPTVDIELVTTTNGEYRIMLSVHGSQHSGMIVNDTGHETDENRAKVEITPNSPRAEATLGGFGSLGLDCEPRTGSGHLKCRLKMDKLRLKG

SEQ ID NO: 2 - protein, 534 aa, Artificial
  Zika EDIII MBP
MKIKTGARILALSALTTMMFSASALAKSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRS

SEQ ID NO: 3 - protein, 534 aa, Artificial
  Zika EDIII variant MBP
MKIKTGARILALSALTTMMFSASALAKSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAGVSYSLCTAAFTFTKHPAETGHGTVQVEVQYAGTDGPCKVPAQMATDLNDLTPVGRLITANPVITESTENSKMMLELDPPFGDSYIVIGVGEKKITHHWHRS

SEQ ID NO: 4 - protein, 436 aa, Artificial
  Zika EDIII Halo-tag
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGIEENLYFQSNAGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSSGGSLPETGGHHHHHH

SEQ ID NO: 5 - protein, 360 aa, Artificial
  Zika NS1 C-terminal His
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGGHHHHHH

SEQ ID NO: 6 - protein, 356 aa, Artificial
  Zika NS1 N-terminal His
GHHHHHHDVGCSVDFSKKETRCGTGVFVYNDVEAWRDRYKYHPDSPRRLAAAVKQAWEDGICSSVSRMNIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 7 - protein, 613 aa, Artificial
  Zika C-terminal NS1 MBP
MKIKTGARILALSALTTMMFSASALAKSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 8 - protein, 102 aa, Artificial
  Zika EDIII domain
GVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRS

SEQ ID NO: 9 - protein, 128 aa, Artificial
  Zika EDI domain
IRCIGVSNRDFVEGMSGGTWVDVVLEHGGCVTVMAQDKPTVDIELVTTTNGEYRIMLSVHGSQHSGMIVNDTGHETDENRAKVEITPNSPRAEATLGGFGSLGLDCEPRTGSGHLKCRLKMDKLRLKG

SEQ ID NO: 10 - protein, 102 aa, Artificial
  Zika EDIII domain variant
GVSYSLCTAAFTFTKHPAETGHGTVQVEVQYAGTDGPCKVPAQMATDLNDLTPVGRLITANPVITESTENSKMMLELDPPFGDSYIVIGVGEKKITHHWHRS

SEQ ID NO: 11 - protein, 181 aa, Artificial
  Zika NS1 C-terminal beta ladder domain
REDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 12 - protein, 349 aa, Artificial
  Zika NS1 polypeptide
DVGCSVDFSKKETRCGTGVFVYNDVEAWRDRYKYHPDSPRRLAAAVKQAWEDGICSSVSRMNIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

(19) United States
(12) Patent Application Publication
(10) Pub. No.: US 2020/0300855 A1
(43) Pub. Date: Sep. 24, 2020

(54) METHODS AND COMPOSITIONS FOR ZIKA VIRUS DETECTION

(71) Applicant: The University of North Carolina at Chapel Hill, Chapel Hill, NC (US)

(72) Inventors: Premkumar Lakshmanane, Chapel Hill, NC (US); Aravinda Desilva, Chapel Hill, NC (US)

(21) Appl. No.: 16/756,703

(22) PCT Filed: Oct. 16, 2018
(86) PCT No.: PCT/US2018/056091

§ 371 Date: Apr. 16, 2020

Related U.S. Application Data
(60) Provisional application No. 62/572,908, filed on Oct. 16, 2017.

(51) Int. Cl. G01N 33/569; C12N 7/00; C07K 14/005

(52) U.S. Cl. CPC G01N 33/56983; C12N 7/00; C12N 2770/24122; C12N 2770/24134; C12N 2770/24123; C07K 14/005

(57) ABSTRACT

The present invention methods and compositions for differentiating a Zika virus infection in a subject from infection by a different flavivirus.

### STATEMENT OF PRIORITY

This application is a 35 U.S.C. § 371 national phase application of International Application Serial No. PCT/US2018/056091, filed Oct. 16, 2018, which claims the benefit, under 35 U.S.C. § 119(e), of U.S. Provisional Application Ser. No. 62/572,908, filed Oct. 16, 2017, the entire contents of each of which are incorporated by reference herein.

### STATEMENT OF GOVERNMENT SUPPORT

This invention was made with government support under Grant Nos. AI107731 and AI134073 awarded by the National Institutes of Health, and Grant No. 200-2017-93142 awarded by the Centers for Disease Control and Prevention. The United States government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-822 ST25.txt, 37,871 bytes in size, generated on May 29, 2020 and filed via EFS-Web, is provided in lieu of a paper copy. This Sequence Listing is hereby incorporated herein by reference into the specification for its disclosures.

### FIELD OF THE INVENTION

The present invention is directed to antigens that distinguish Zika virus infection from other flavivirus infections.

### BACKGROUND OF THE INVENTION

Zika virus (ZIKV) is an emerging flavivirus that can cause birth defects and neurologic complication. Molecular tests are effective in diagnosing acute ZIKV infection, although the majority of infections produce no symptoms at all or present after the narrow window in which molecular diagnostics are dependable. Serology is a reliable method for detecting infections after the viremic period; however, most serological assays have limited specificity due to cross-reactive antibodies elicited by flavivirus infections. Since ZIKV and dengue virus (DENV) widely co-circulate, distinguishing Zika from dengue virus infection is particularly important for diagnosing individual cases or surveillance to coordinate public health response. Flaviviruses also elicit type-specific antibodies directed to non-cross-reactive epitopes of the infecting virus and such epitopes can be used as targets for designing antigens to develop serologic tests with greater specificity.

The present invention overcomes previous shortcomings in the art by providing compositions and methods directed to antibodies and epitopes for use in diagnostics and vaccines directed to Zika virus.

### BRIEF DESCRIPTION OF THE DRAWINGS

FIG. 1. Identification of envelope protein antigens for serodiagnosis of ZIKV. Mapping of type-specific (Panel A) and cross-reactive (Panel B) epitopes on E protein was performed by using experimentally determined antibody complex structures. Footprints of antibody on E protein are shown as spheres. (Panel C) Surface amino acid conservation among clinically relevant flaviviruses. Three highly variable regions that overlap type-specific antibody-binding regions in panel A were identified as putative ZIKV-specific antibody-binding regions, and the corresponding amino acid residues within this region are shown as spheres.

FIG. 2. Analysis of purified recombinant ZIKV antigens by SDS-PAGE and size exclusion chromatography (SEC). Purified Z-EDI (Panel A) and Z-EDIII (Panel B) antigens (6 μg) were subjected to SDS-PAGE under reducing conditions and then stained with Coomassie brilliant blue. Molecular size markers and their apparent masses are shown on the left. (Panel C) SEC overlays of purified Z-EDI, Z-EDIII, and MBP antigens. Protein samples in PBS were subjected to SEC on a Superdex75 10/300GL column. mAU, milli-absorbance units.

FIG. 3: Dotplot graphs showing IgG ELISA binding of recombinant ectodomain of envelope protein (E80) antigen to sera from patients with remote (Panel A) and recent (Panel B) ZIKV infections. Primary and secondary serum samples from ZIKV and DENV infected patients were diluted to 1:20 and the IgG antibodies bound to recombinant E80 antigen were measured using standard ELISA. Statistical significances are indicated at the top (Mann-Whitney U-test). A p value of <0.0001 was considered statistically significant. The horizontal lines represent the means.

FIG. 4: Dotplot graphs showing IgG sandwich ELISA binding of Z-EDI and Z-EDIII with remote (Panels A and B) and recent (Panels C and D) convalescent sera from patients infected with ZIKV, and/or DENV. Primary (grey) and secondary (black) human serum samples were diluted to 1:20 and the bound IgG antibodies to Z-EDI (Panels A and C) or Z-EDIII (Panels B and D) was measured using sandwich ELISA. Statistical significances are indicated at the top (Mann-Whitney U-test). A p value of <0.0001 was considered statistically significant. The horizontal lines represent the means.

FIG. 5: Effect of serum dilution on the specificity of EDIII reactivity with early convalescent sera from patients infected with ZIKV, and/or DENY. Primary (grey) and secondary (black) human serum samples were diluted at 1:20, 1:60 or 1:180 and the bound IgG Abs to Z-EDIII was measured using sandwich ELISA as described in the methods section.

FIG. 6. Longitudinal analysis of Ab response to Z-EDIII. PCR confirmed ZIKV immune serum samples collected between 1- and 190-days post-infection were analyzed by Z-EDIII ELISA. Z-EDIII ELISA results showed that samples collected after first week post ZIKV infection can be positively identified.

FIG. 7. Sensitivity of Z-EDIII ELISA for detection of acute and remote ZIKV infection in DENV naïve and DENV immune people. Sequential ZIKV immune serum samples collected around 28 days and 180 days post-infection from DENV naïve and DENV immune patients were analyzed by Z-EDIII ELISA. Z-EDIII ELISA showed >95% sensitivity for detecting both acute (29/29) and remote (28/29) ZIKV infection in DENV naïve and DENV immune patients.

FIG. 8. Binding of recombinant Z-E80, Z-EDI and Z-EDIII with convalescent-phase sera collected >12 weeks post-infection from patients infected with ZIKV and/or DENV. DENV-naïve (filled symbols) and DENV immune (unfilled symbols). Human serum samples were diluted 1:20, and the IgG antibodies bound to E80, Z-EDI or Z-EDIII were measured using ELISA assay. Sera collected 12 weeks after infection were defined as remote infections, and sera collected within the first 12 weeks were considered to represent recent infections. Statistical significances are indicated at the top of the graphs (Mann-Whitney U test). P values of 0.0001 were considered statistically significant. The horizontal lines represent the means. Z-EDIII, and to a lesser extent by Z-EDI, can distinguish remote ZIKV infection from DENY infections.

FIG. 9. Binding of recombinant Z-EDIII with recent convalescent-phase sera collected <12 weeks post-infection from patients infected with ZIKV and/or DENV. DENY-naïve (filled symbols) and DENY immune (unfilled symbols). Human serum samples were diluted 1:20, and the IgG antibodies bound to Z-EDIII were measured using ELISA assay. Sera collected 12 weeks after infection were defined as remote infections, and sera collected within the first 12 weeks were considered to represent recent infections. The horizontal lines represent the means. Z-EDIII showed some level of cross-reactivity when serum samples were collected from individuals experiencing recent secondary DENV infection or primary DENV1-immune.

FIG. 10. Schematics of the assay steps in an improved Z-EDIII sandwich ELISA. The earlier Z-EDIII sandwich ELISA relied on mouse anti-MBP capture antibody. This assay set-up caused background issues with some serum samples due to heterophilic antibody interference with mouse capture antibody. Also, this assay format did not permit analyzing mouse serum samples as capture antibody used was of mouse origin (anti-MBP). To eliminate the background issues, as well as to reduce Z-EDIII cross-reactivity with recent secondary DENV immune sera by antigen competition, the Z-EDIII assay based on antibody-MBP-EDIII capture was reformatted with a streptavidin-biotinylated EDIII capture step.

FIG. 11. SDS-PAGE analysis of purity and biotinylation of Halo-tag fused Z-EDIII. (Panel A) Affinity purified Halo-tag fused Z-EDIII antigen (6 μg) produced in mammalian cells was subjected to SDS-PAGE under reducing conditions and then stained with Coomassie brilliant blue. Molecular size markers and their apparent masses are shown on the left. (Panel B) Gel-shift analysis of site-specifically biotin labeled Halo-tag fused Z-EDIII. Biotin labeled Z-EDIII was mixed with streptavidin and subjected to non-reducing SDS-PAGE with non-boiled samples.

FIG. 12. Evaluation of improved Z-EDIII ELISA. Binding analysis of biotin labeled Z-EDIII with ZIKV and DENV immune sera on streptavidin coated ELISA plate.

FIG. 13. Surface amino acid conservation of NS1 antigen among clinically relevant flaviviruses. The β-ladder domain was identified as highly conserved across flaviviruses and considered to represent cross-reactive antibody-binding surface in NS1 antigen.

FIG. 14. Schematics of the assay steps in Z-NS1 antigen competition ELISA. A competitive NS1 ELISA (Z-NS1 cELISA) was developed, which pits between plate-immobilized NS1 and Z-NS1 β-ladder domain in solution for binding to Z-NS1 specific Ab. In step A, full length Z-NS1 is coated on to ELISA microtiter plate. In step B, pre-incubated serum with Z-NS1 β-ladder domain is added to the microtiter plate coated with Z-NS1 Ag. In step C, the wells are washed to remove unbound serum antibody, antibody complexes and soluble Z-NS1 β-ladder domain. In step D, labeled secondary antibody is added to measure binding of Z-NS1 specific IgG.

FIG. 15. Sensitivity of Z-NS1 antigen competition ELISA (Z-NS1 cELISA) for detection of acute and remote ZIKV infection in DENV naïve and DENV immune people. Sequential ZIKV immune serum samples collected around 28 days and 180 days post-infection from DENV naïve and DENV immune patients were analyzed by Z-NS1 cELISA. Z-NS1 cELISA showed >95% sensitivity for detecting both acute (29/29) and remote (28/29) ZIKV infection in DENV naïve and DENV immune patients.

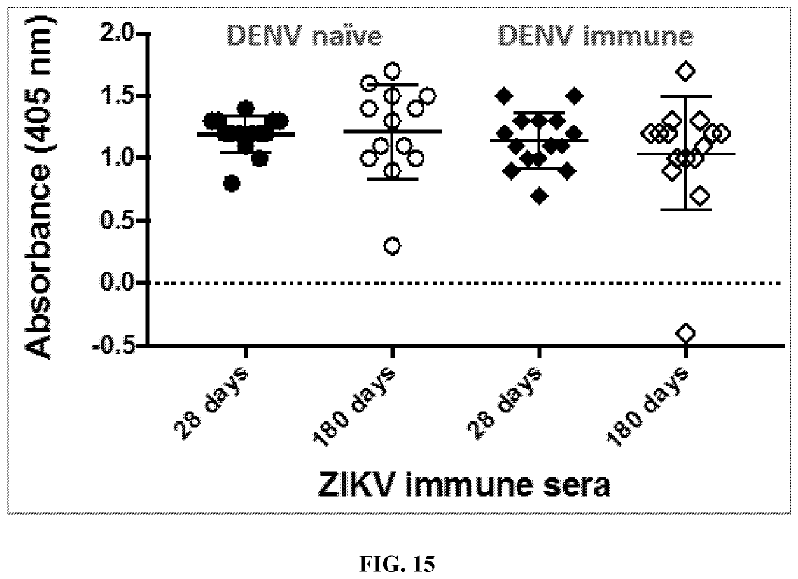

FIG. 16. Specificity of Z-NS1 cELISA against early convalescent secondary DENV immune sera. Thirty secondary DENV immune sera collected between 2- and 9-weeks post-infection were analyzed by Z-NS1 direct ELISA and Z-NS1 cELISA. Specificity of FL-NS1 direct ELISA was less than 50%, whereas Z-NS1 cELISA specificity was 93% (i.e. 28 of 30 samples were identified correctly as true negative by Z-NS1 cELISA).

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides a recombinant polypeptide comprising a Zika EDI, EDIII, or NS1 domain or polypeptide fused to a maltose binding protein or Halo Tag or His tag.

In a further embodiment, the present invention provides a nucleic acid molecule encoding the recombinant polypeptide of this invention.

The present invention also provides a method of diagnosing a Zika virus infection in a subject, comprising: a) contacting a sample from the subject with the recombinant polypeptide of this invention under conditions whereby an antigen/antibody complex can form; and b) detecting formation of the antigen/antibody complex, thereby diagnosing a Zika virus infection in the subject.

Additionally provided herein is a method of detecting an antibody to Zika virus in a sample, comprising: a) contacting the sample with the recombinant polypeptide of this invention under conditions whereby an antigen/antibody complex can form; and b) detecting formation of the antigen/antibody complex, thereby detecting an antibody to Zika virus in the sample.

Further provided herein is a method of identifying an infection by Zika virus in a subject known to have, or suspected of having, a flavivirus infection, comprising a) contacting a sample from the subject with the recombinant polypeptide of this invention under conditions whereby an antigen/antibody complex can form; and b) detecting formation of the antigen/antibody complex, thereby identifying an infection by Zika virus in the subject.

In one embodiment, the present invention provides a method of identifying an infection by Zika virus in a subject, comprising: a) contacting a serum sample from the subject with a recombinant Z-NS1 β-ladder domain under conditions whereby an antigen/antibody complex can form; b) contacting the serum sample comprising the recombinant Z-NS1 β-ladder domain of step (a) with a full length Zika NS1 polypeptide that is bound to a solid substrate in a reaction well under conditions whereby an antigen/antibody complex can form; c) washing the reaction well of (b) to remove unbound antibody, unbound antigen/antibody complexes, and unbound recombinant Zika NS1 (3-ladder domain; and d) detecting the formation of an antigen/antibody complex comprising the full length Zika NS1 polypeptide bound to the solid substrate, thereby identifying an infection by Zika virus in the subject.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” (and grammatical variants) means that the scope of a claim is to be interpreted to encompass the specified materials or steps recited in the claim, “and those that do not materially affect the basic and novel characteristic(s)” of the claimed invention. Thus, the term “consisting essentially of” when used in a claim of this invention is not intended to be interpreted to be equivalent to “comprising.”

The present invention is based on the unexpected discovery of antigens that allow for the identification of Zika virus infection in a subject and for distinguishing Zika virus infection from other flavivirus infection.

Thus, in one embodiment, the present invention provides a recombinant polypeptide comprising a Zika EDI domain, a Zika EDIII domain, a Zika NS1 polypeptide, a Zika NS1 ladder domain, and/or a Zika EDIII domain variant fused to a fusion binding protein such as a recombinant protein fusion partner and/or other linking protein partner. A recombinant protein fusion partner or linking protein partner of this invention can be linked to a Zika EDI domain or a Zika EDIII domain or a Zika NS1 domain or a Zika NS1 full length polypeptide of this invention by translation of a nucleotide sequence encoding the fusion partner in frame with the domain or polypeptide and/or by covalently linking or joining the domain or polypeptide to the protein or peptide fusion partner or linking protein partner to which the domain is to be linked or joined.

Nonlimiting examples of a protein fusion partner or linking protein partner of this invention include maltose binding protein (MBP), glutathione S transferase (GST), green fluorescent protein (GFP), thioredoxin, NusA, calmodulin binding peptide, Fhb fusion system, Fc domain of immunoglobulin, synthetic oligomerization scaffolding protein, DsBa, DsBc, Mistic, Sortase, small ubiquitin-like modifier (Sumo), SpyTag peptide, SNAP protein labeling system, CLIP protein labeling system, Histidine affinity tag (His tag), and mutated hydrolase such as HALOTAG® protein, as well as any other fusion protein or linking protein now known or later identified. These protein fusion partners and linking protein partners can be used singly or in any combination and/or in any multiples.

Thus, in particular embodiments, the present invention provides a recombinant polypeptide, e.g., for use in an immunoassay, comprising an amino acid sequence selected from the group consisting of:

a)
(SEQ ID NO: 1)

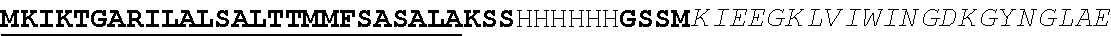

VGKKFEKDTGIKVTVEHPDKLEEKFPQVAATGDGPDIIFWAHDRFGGYAQSGLLAEITPDKAFQDKLYPFT
WDAVRYNGKLIAYPIAVEALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKGETAMTINGPWAWSNIDTS
KVNYGVTVLPTFKGQPSKPFVGVLSAGINAASPNKELAKEFLENYLLTDEGLEAVNKDKPLGAVALKSYEEEL

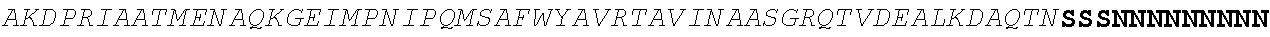

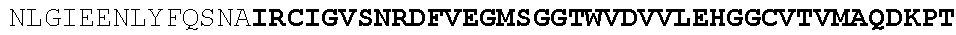

VDIELVTTTNGEYRIMLSVHGSQHSGMIVNDTGHETDENRAKVEITPNSPRAEAT
LGGFGSLGLDCEPRTGSGHLKCRLKMDKLRLKG (EDI-MBP);
b)
(SEQ ID NO: 2)

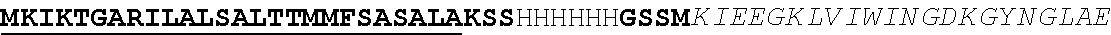

VGKKFEKDTGIKVTVEHPDKLEEKFPQVAATGDGPDIIFWAHDRFGGYAQSGLLAEITPDKAFQDKLYPFT
WDAVRYNGKLIAYPIAVEALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKGETAMTINGPWAWSNIDTS
KVNYGVTVLPTFKGQPSKPFVGVLSAGINAASPNKELAKEFLENYLLTDEGLEAVNKDKPLGAVALKSYEEEL

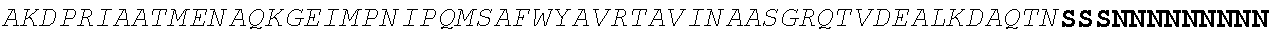

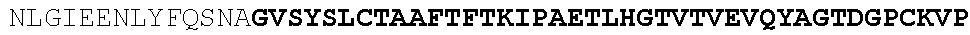

AQMAVDMQTLTPVGRLITANPVITESTENSKMMLELDPPFGDSYIVIGVGEKKIT
HHWHRS (EDIII-MBP);
c)
(SEQ ID NO: 3)

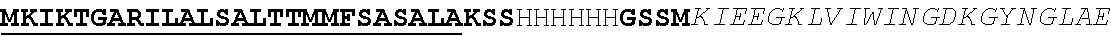

VGKKFEKDTGIKVTVEHPDKLEEKFPQVAATGDGPDIIFWAHDRFGGYAQSGLLAEITPDKAFQDKLYPFT
WDAVRYNGKLIAYPIAVEALSLIYNKDLLPNPPKTWEEIPALDKELKAKGKSALMFNLQEPYFTWPLIAADGG
YAFKYENGKYDIKDVGVDNAGAKAGLTFLVDLIKNKHMNADTDYSIAEAAFNKGETAMTINGPWAWSNIDTS
KVNYGVTVLPTFKGQPSKPFVGVLSAGINAASPNKELAKEFLENYLLTDEGLEAVNKDKPLGAVALKSYEEEL

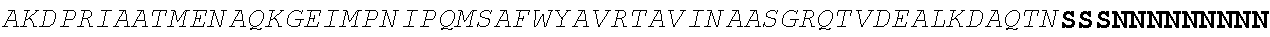

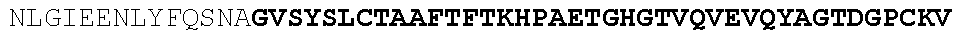

PAQMATDLNDLTPVGRLITANPVITESTENSKMMLELDPPFGDSYIVIGVGEKKI
THHWHRS (EDIII variant-MBP);
d)
(SEQ ID NO: 4)
MKWVTFISLLFLFSSAYSMAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLEEVVLVIHDWGSALGFHWAKRNPE
RVKGIAFMEFIRPIPTWDEWPEFARETFQAFRTTDVGRKLIIDQNVFIEGTLPMGVVRPLTEVEMDHYREPFL
NPVDREPLWRFPNELPIAGEPANIVALVEEYMDWLHQSPVPKLLFWGTPGVLIPPAEAARLAKSLPNCKAVDI

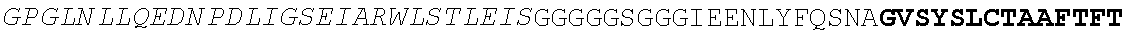

KIPAETLHGTVTVEVQYAGTDGPCKVPAQMAVDMQTLTPVGRLITANPVITEST

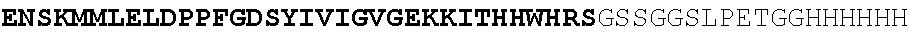

(EDIII-Halo tag);
e)
(SEQ ID NO: 5)
MYRMQLLSCIALSLALVTNSDVGCSVDFSKKETRCGTGVFVYNDVEAWRDRYKY
HPDSPRRLAAAVKQAWEDGICGISSVSRMENIMWRSVEGELNAILEENGVQLTV
VVGSVKNPMWRGPQRLPVPVNELPHGWKAGKSYFVRAAKTNNSFVVDGDTL
KECPLKHRAWNSFLVEDHGFGVFHTSVWLKVREDYSLECDPAVIGTAVKGKEA
VHSDLGYWIESEKNDTWRLKRAHLIEMKTCEWPKSHTLWTDGIEESDLIIPKSL
AGPLSHHNTREGYRTQMKGPWHSEELEIRFEECPGTKVHVEETCGTRGPSLRS

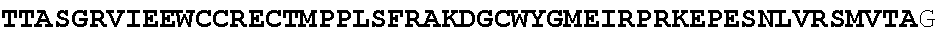

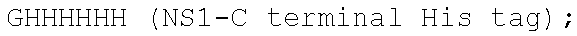

f)
(SEQ ID NO: 6)

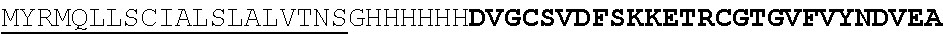

WRDRYKYHPDSPRRLAAAVKQAWEDGICSSVSRMNIMWRSVEGELNAILEENG
VQLTVVVGSVKNPMWRGPQRLPVPVNELPHGWKAWGKSYFVRAAKTNNSFVV
DGDTLKECPLKHRAWNSFLVEDHGFGVFHTSVWLKVREDYSLECDPAVIGTAV
KGKEAVHSDLGYWIESEKNDTWRLKRAHLIEMKTCEWPKSHTLWTDGIEESDL
IIPKSLAGPLSHHNTREGYRTQMKGPWHSEELEIRFEECPGTKVHVEETCGTRG
PSLRSTTASGRVIEEWCCRECTMPPLSFRAKDGCWYGMEIRPRKEPESNLVRSM
VTA (NS1-N terminal His tag);
g)
(SEQ ID NO: 7)

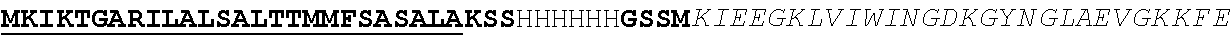

KDTGIKVTVEHPDKLEEKFPQVAATGDGPDIIFWAHDRFGGYAQSGLLAEITPDKAFQDKL
YPFTWDAVRYNGKLIAY
PIAVEALSLIYNKDLLPNPPKTWEEIPALDKELKAKGKSALMFNLQEPYFTWPLIAADGG
YAFKYENGKYDIKDVGVDN
AGAKAGLTFLVDLIKNKHMNADTDYSIAEAAFNKGETAMTINGPWAWSNIDTSKVNYGVTVL
PTFKGQPSKPFVGVLS
AGINAASPNKELAKEFLENYLLTDEGLEAVNKDKPLGAVALKSYEEELAKDPRIAAT
MENAQKGEIMPNIPQMSAFWYA

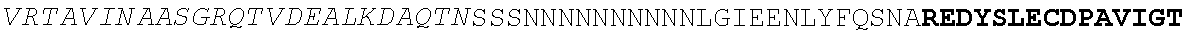

AVKGKEAVHSDLGYWIESEKNDTWRLKRAHLIEMKTCEWPKSHTLWTDGIEESDLIIP
KSLAGPLSHHNTREGYRTQMKGPWHSEELEIRFEECPGTKVHVEETCGTRGPSLRSTT
ASGRVIEEWCCRECTMPPLSFRAKDGCWYGMEIRPRKEPESNLVRSMVTA (C-terminal
NS1-MBP); and
h) any combination of (a)-(g).

In the above sequences (a) through (g) above, the underlined text identifies a signal peptide; grey text identifies a His-affinity tag; italic text identifies a protein fusion partner or linking protein partner (e.g., MBP/Halo); the grey highlighted text identifies a linker amino acid sequence; and the bolded text identifies the amino acid sequence of the antigen (e.g., EDI/EDIII/NS1).

One nonlimiting example of a recombinant polypeptide comprising a Zika EDI domain fused to a maltose binding protein is an amino acid sequence comprising, consisting essentially of or consisting of the amino acid sequence (Zika EDI domain is bolded):

(SEQ ID NO: 1)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNGIEENLYFQSNAIRCIGVSNRDFVEGMSGGT
WVDVVLEHGGCVTVMAQDKPTVDIELVTTTNGEYRIMLSVHGSQHSGMIV
NDTGHETDENRAKVEITPNSPRAEATLGGFGSLGLDCEPRTGSGHLKCRL
KMDKLRLKG.

In a further embodiment, the present invention provides a recombinant polypeptide comprising a Zika EDIII domain fused to a fusion binding protein.

One nonlimiting example of a recombinant polypeptide comprising a Zika EDIII domain fused to a maltose binding protein is an amino acid sequence comprising, consisting essentially of or consisting of the amino acid sequence Zika EDIII domain is bolded):

(SEQ ID NO: 2)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNGIEENLYFQSNAGVSYSLCTAAFTFTKIPAE
TLHGTVTVEVQYAGTDGPCKVPAQMAVDMQTLTPVGRLITANPVITESTE
NSKMMLELDPPFGDSYIVIGVGEKKITHHWHRS.

One nonlimiting example of a recombinant polypeptide comprising a Zika EDIII domain fused to a Halo tag is an amino acid sequence comprising, consisting essentially of or consisting of the amino acid sequence Zika EDIII domain is bolded):

(SEQ ID NO: 4)
MKWVTFISLLFLFSSAYSMAEIGTGFPFDPHYVEVLGERMHYVDVGPRDG
TPVLFLHGNPTSSYVWRNIIPHVAPTHRCIAPDLIGMGKSDKPDLGYFFD
DHVRFMDAFIEALGLEEVVLVIHDWGSALGFHWAKRNPERVKGIAFMEFI
RPIPTWDEWPEFARETFQAFRTTDVGRKLIIDQNVFIEGTLPMGVVRPLT
EVEMDHYREPFLNPVDREPLWRFPNELPIAGEPANIVALVEEYMDWLHQS
PVPKLLFWGTPGVLIPPAEAARLAKSLPNCKAVDIGPGLNLLQEDNPDLI
GSEIARWLSTLEISGGGGGSGGGIEENLYFQSNAGVSYSLCTAAFTFTKI
PAETLHGTVTVEVQYAGTDGPCKVPAQMAVDMQTLTPVGRLITANPVITE
STENSKMMLELDPPFGDSYIVIGVGEKKITHHWHRSGSSGGSLPETGGHH
HHHH (EDIII-Halo tag).

One nonlimiting example of a recombinant polypeptide comprising a Zika EDIII domain variant fused to a maltose binding protein is an amino acid sequence comprising, consisting essentially of or consisting of the amino acid sequence (Zika EDIII domain variant is bolded):

(SEQ ID NO: 3)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNGIEENLYFQSNAGVSYSLCTAA
FTFTKHPAET GHGTVQVEVQ YAGTDGPCKV PAQMATDLND
LTPVGRLITA NPVITESTEN SKMMLELDPP FGDSYIVIGV
GEKKITHHWH RS.

This variant has been produced by introducing the following substitutions: I16H; L21G; T26Q; V46T; M48L; Q49N; and T50D into the amino acid sequence of the Zika EDIII domain, having the amino acid sequence GVSYSLCTAA FTFTKIPAET LHGTVTVEVQ YAGTDGPCKV PAQMAVDMQT LTPVGRLITA NPVITESTEN SKMMLELDPP FGDSYIVIGV GEKKITHHWH RS (SEQ ID NO:8), and numbering of the residues is based on the numbering of the amino acid residues in this sequence EDIII domain sequence.

Additional Zika EDIII domain variants can be made by introducing one or more (e.g., 2, 3, 4, 5, 6, 7, 8, 9, 10, 11, 12, 13, 14, 15, 16, 17, 18, 19, 20, etc.) substituted amino acid residues in the Zika EDIII domain sequence at any position and in any combination. Variants of this invention can also be made by insertion of amino acid residues and/or deletion of amino acid residues, with or without substitution of original amino acids residues. Amino acid residues that can be substituted include naturally occurring amino acid residues such as those shown in Table 3, as well as any modified amino acid residues such as those shown in Table 4.

One nonlimiting example of a recombinant polypeptide comprising a Zika NS1 polypeptide comprising a C-terminal His tag is an amino acid sequence comprising, consisting essentially of or consisting of the amino acid sequence (Zika NS1 polypeptide is bolded):

(SEQ ID NO: 5)
MYRMQLLSCIALSLALVTNSDVGCSVDFSKKETRCGTGVFVYNDVEAWRD
RYKYHPDSPRRLAAAVKQAWEDGICGISSVSRMENIMWRSVEGELNAILE
ENGVQLTVVVGSVKNPMWRGPQRLPVPVNELPHGWKAWGKSYFVRAAKTN
NSFVVDGDTLKECPLKHRAWNSFLVEDHGFGVFHTSVWLKVREDYSLECD
PAVIGTAVKGKEAVHSDLGYWIESEKNDTWRLKRAHLIEMKTCEWPKSHT
LWTDGIEESDLIIPKSLAGPLSHHNTREGYRTQMKGPWHSEELEIRFEEC
PGTKVHVEETCGTRGPSLRSTTASGRVIEEWCCRECTMPPLSFRAKDGCW
YGMEIRPRKEPESNLVRSMVTAGGHHHHHH (NS1-C terminal
His tag).

One nonlimiting example of a recombinant polypeptide comprising a Zika NS1 polypeptide comprising an N-terminal His tag is an amino acid sequence comprising, consisting essentially of or consisting of the amino acid sequence (Zika NS1 polypeptide is bolded):

(SEQ ID NO: 6)
MYRMQLLSCIALSLALVTNSGHHHHHHDVGCSVDFSKKETRCGTGVFVYN
DVEAWRDRYKYHPDSPRRLAAAVKQAWEDGICSSVSRMNIMWRSVEGELN
AILEENGVQLTVVVGSVKNPMWRGPQRLPVPVNELPHGWKAWGKSYFVRA
AKTNNSFVVDGDTLKECPLKHRAWNSFLVEDHGFGVFHTSVWLKVREDYS
LECDPAVIGTAVKGKEAVHSDLGYWIESEKNDTWRLKRAHLIEMKTCEWP
KSHTLWTDGIEESDLIIPKSLAGPLSHHNTREGYRTQMKGPWHSEELEIR
FEECPGTKVHVEETCGTRGPSLRSTTASGRVIEEWCCRECTMPPLSFRAK
DGCWYGMEIRPRKEPESNLVRSMVTA (NS1-N terminal
His tag).

One nonlimiting example of a recombinant polypeptide comprising a Zika NS1 C-terminal β ladder domain fused to a maltose binding protein is an amino acid sequence comprising, consisting essentially of or consisting of the amino acid sequence (Zika NS1 ladder domain is bolded):

(SEQ ID NO: 7)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNGIEENLYFQSNAREDYSLECDPAVIGTAVKG
KEAVHSDLGYWIESEKNDTWRLKRAHLIEMKTCEWPKSHTLWTDGIEESD
LIIPKSLAGPLSHHNTREGYRTQMKGPWHSEELEIRFEECPGTKVHVEET
CGTRGPSLRSTTASGRVIEEWCCRECTMPPLSFRAKDGCWYGMEIRPRKE
PESNLVRSMVTA (C-terminal NS1-MBP).

Additional embodiments of this invention include Zika EDI domain, Zika EDIII domain, Zika NS1 C-terminal β ladder domain and Zika NS1 full length polypeptide alone without any tags or fusion or linker partners and/or with an MBP fusion partner, a C-terminal His affinity tag, an N-terminal His affinity tag, a Halo tag or any other tag or fusion partner, in any combination and in any multiplicity. For example, in an immunoassay of this invention, one or more antigens may have no tag or fusion partner; one or more antigens may have MBP as a fusion partner; one or more antigens may have a C-terminal His affinity tag, one or more antigens may have an N-terminal His affinity tag, one or more antigens may have a Halo tag, and/or one or more antigens may have any other tag or fusion partner, in any combination.

It is also contemplated that any of the amino acid sequences of this invention can include or omit the signal peptide and/or the linker amino acid sequence, and/or the affinity tag and/or the fusion partner or linking protein partner, in any combination.

As used herein, the term “amino acid” or “amino acid residue” encompasses any naturally occurring amino acid, modified forms thereof, and synthetic amino acids.

Naturally occurring, levorotatory (L-) amino acids are shown in Table 4). Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described, e.g., by Wang et al. Annu Rev Biophys Biomol Struct. 35:225-49 (2006)). These unnatural amino acids can advantageously be used to chemically link molecules of interest to the polypeptide of this invention.

The present invention additionally provides a nucleic acid molecule encoding the recombinant polypeptide of this invention.

Also provided herein is a composition comprising the recombinant polypeptide of this invention and/or the nucleic acid molecule of this invention in a pharmaceutically acceptable carrier.

Nonlimiting examples of the EDI and/or EDIII domains and/or NS1 domains of this invention that can be included in a composition of this invention include:

EDI domain:
(SEQ ID NO: 9)
IRCIGVSNRDFVEGMSGGTWVDVVLEHGGCVTVMAQDKPTVDIELVTTTN
GEYRIMLSVHGSQHSGMIVNDTGHETDENRAKVEITPNSPRAEATLGGFG
SLGLDCEPRTGSGHLKCRLKMDKLRLKG.
EDIII domain:
(SEQ ID NO: 8)
GVSYSLCTAAFTFTKIPAETLHGTVTVEVQYAGTDGPCKVPAQMAVDMQT
LTPVGRLITANPVITESTENSKMMLELDPPFGDSYIVIGVGEKKITHHWH
RS.
EDIII domain variant:
(SEQ ID NO: 10)
GVSYSLCTAA FTFTKHPAET GHGTVQVEVQ YAGTDGPCKV
PAQMATDLND LTPVGRLITA NPVITESTEN SKMMLELDPP
FGDSYIVIGV GEKKITHHWH RS.
NS1 C-terminal β ladder domain:
(SEQ ID NO: 11)
REDYSLECDPAVIGTAVKGKEAVHSDLGYWIESEKNDTWRLKRAHLIEMK
TCEWPKSHTLWTDGIEESDLIIPKSLAGPLSHHNTREGYRTQMKGPWHSE
ELEIRFEECPGTKVHVEETCGTRGPSLRSTTASGRVIEEWCCRECTMPPL
SFRAKDGCWYGMEIRPRKEPESNLVRSMVTA.
NS1 polypeptide:
(SEQ ID NO: 12)
DVGCSVDFSKKETRCGTGVFVYNDVEAWRDRYKYHPDSPRRLAAAVKQAW
EDGICSSVSRMNIMWRSVEGELNAILEENGVQLTVVVGSVKNPMWRGPQR
LPVPVNELPHGWKAWGKSYFVRAAKTNNSFVVDGDTLKECPLKHRAWNSF
LVEDHGFGVFHTSVWLKVREDYSLECDPAVIGTAVKGKEAVHSDLGYWIE
SEKNDTWRLKRAHLIEMKTCEWPKSHTLWTDGIEESDLIIPKSLAGPLSH
HNTREGYRTQMKGPWHSEELEIRFEECPGTKVHVEETCGTRGPSLRSTTA
SGRVIEEWCCRECTMPPLSFRAKDGCWYGMEIRPRKEPESNLVRSMVTA.

It is contemplated that in some embodiments, the recombinant polypeptides and/or nucleic acid molecules of this invention can be used as immunogens and/or in a vaccine formulation. In particular, Zika EDI and/or Zika EDIII and/or NS1 domains of this invention with or without a protein fusion partner can be included in a pharmaceutical formulation, in any combination and/or ratio relative to one another.

Thus, the present invention further provides a method of inducing an immune response in a subject, comprising administering to the subject an effective amount of a Zika EDI and/or Zika EDIII domain of this invention.

Also provided herein is a method of reducing or protecting against the effects of infection by Zika virus in a subject (e.g., a subject in need thereof), comprising administering to the subject an effective amount of a Zika EDI and/or Zika EDIII domain of this invention. A subject in need thereof can be a subject at risk of having Zika virus infection or at risk of being infected by Zika virus. In some embodiments, the subject is a female who is pregnant or planning to become pregnant or has the potential to become pregnant. In some embodiments, the subject is a fetus. In some embodiments, the subject can be a male sex partner of a female who is pregnant or trying to become pregnant or has the potential to become pregnant.

In some embodiments, the recombinant polypeptide or peptide of this invention can be linked or bound to a solid substrate (e.g., in a reaction vessel or well, which can be a well of a microtiter plate) and in some embodiments, the recombinant polypeptide of this invention can be linked to a detectable moiety.

In some embodiments, the recombinant polypeptide or peptide of this invention can be linked to a Halo Tag and a biotin molecule can be incorporated into the Halo Tag so that the recombinant polypeptide or peptide can be bound by streptavidin.

The recombinant polypeptides of this invention can be used in various methods. In one embodiment, the present invention provides a method of diagnosing a Zika virus infection in a subject, comprising: a) contacting a sample from the subject with the recombinant polypeptide of this invention under conditions whereby an antigen/antibody complex can form; and b) detecting formation of the antigen/antibody complex, thereby diagnosing a Zika virus infection in the subject.

In further embodiments, the present invention provides a method of detecting an antibody to Zika virus in a sample, comprising: a) contacting the sample with the recombinant polypeptide of this invention under conditions whereby an antigen/antibody complex can form; and b) detecting formation of the antigen/antibody complex, thereby detecting an antibody to Zika virus in the sample.

Additionally provided herein is a method of identifying an infection by Zika virus in a subject known to have, or suspected of having, a flavivirus infection, comprising: a) contacting a sample from the subject with the recombinant polypeptide of this invention under conditions whereby an antigen/antibody complex can form; and b) detecting formation of the antigen/antibody complex, thereby identifying an infection by Zika virus in the subject. Such a method allows for the differentiation between infection by a flavivirus other than Zika (e.g., a dengue virus) and infection by Zika virus. Such differentiation can guide treatment and prophylaxis in a subject and in a population.

In some embodiments, the methods of this invention can be carried out in an immunoassay. Nonlimiting examples of an immunoassay of this invention include an enzyme linked immunosorbent assay (ELISA), a lateral flow assay (LFA) and a multiplex assay. Nonlimiting examples of a multiplex assay of this invention include a plasmonic gold platform and a microbead-based assay.

In one embodiment, the present invention provides a method of identifying an infection by Zika virus in a subject, comprising: a) contacting a serum sample from the subject with a recombinant Z-NS1 β-ladder domain under conditions whereby an antigen/antibody complex can form; b) contacting the serum sample comprising the recombinant Z-NS1 β-ladder domain of step (a) with a full length Zika NS1 polypeptide that is bound to a solid substrate in a reaction well under conditions whereby an antigen/antibody complex can form; c) washing the reaction well of (b) to remove unbound antibody, unbound antigen/antibody complexes, and unbound recombinant Zika NS1 (3-ladder domain; and d) detecting the formation of an antigen/antibody complex comprising the full length Zika NS1 polypeptide bound to the solid substrate, thereby identifying an infection by Zika virus in the subject.

In the methods described herein a subject can be a subject known to have or suspected of having a viral infection, which can be a flavivirus infection. In some embodiments of this invention, the methods described herein can be used to identify a flavivirus infection as a Zika virus infection. The methods described herein can be used to improve the specificity of Zika virus infection detection.

In the methods described herein, the recombinant Zika NS1 (Z-NS1) β ladder domain can comprise the amino acid sequence of SEQ ID NO:7. In some embodiments, the Zika NS1 β ladder domain can be linked to a tag or fusion partner and in some embodiments, the Zika NS1 β ladder domain is not linked to a tag or fusion partner.

In the methods described herein, the full length Zika NS1 polypeptide can comprise the amino acid sequence of SEQ ID NO:5 or SEQ ID NO:6. In some embodiments, the Zika NS1 polypeptide can be linked to a tag or fusion partner and in some embodiments, the Zika NS1 polypeptide is not linked to a tag or fusion partner.

In the methods described herein, the EDIII domain can comprise the amino acid sequence of SEQ ID NO:2, SEQ ID NO:3, or SEQ ID NO:4. In some embodiments, the EDIII domain can be linked to a tag or fusion partner and in some embodiments, the EDIII domain is not linked to a tag or fusion partner.

In the methods described herein, the EDI domain can comprise the amino acid sequence of SEQ ID NO:1. In some embodiments, the EDI domain can be linked to a tag or fusion partner and in some embodiments, the EDI domain is not linked to a tag or fusion partner.

Zika virus is a flavivirus. A nonlimiting example of a Zika virus includes ZIKV-FP2013 (GenBank Accession No. KJ776791.2).

Tamana bat virus (TABV) is a flavivirus that has no known vector and has limited cross-reactivity for antibodies elicited by other flaviviruses. A nonlimiting example of a Tamana bat virus has the amino acid sequence as provided under GenBank Accession No. NC_003996.

There are four serotypes of dengue virus (DENV1, DENV2, DENV3 and DENV4). Within each serotype there are a number of different strains or genotypes. The dengue virus antigens and epitopes of the invention can be derived from any dengue virus, including all serotypes, strains and genotypes, now known or later identified. Nonlimiting examples of dengue viruses of this invention include DENV1 (GenBank Accession No. U88535.1), DENV2 (GenBank Accession No. GU289914.1), DENV3 (GenBank Accession No. JQ411814.1), and DENV4 (GenBank Accession No. KJ160504.1).

Nonlimiting examples of dengue virus include UNC1017 strain (DENV-1), West Pacific 74 strain (DENV-1), 516803 strain (DEN2), UNC2005 strain (DENV-2), UNC3001 strain (DENV-3), UNC3043 (DENV-3 strain 059.AP-2 from Philippines, 1984), UNC3009 strain (DENV-3, D2863, Sri Lanka 1989), UNC3066 (DEN3, strain 1342 from Puerto Rico 1977), CH53489 strain (DENV-3), UNC4019 strain (DENV-4), and TVP-360 (DENV-4).

Nonlimiting examples of other flaviviruses include yellow fever virus (YFV) (e.g., GenBank® Database Accession No. JX503529) Japanese encephalitis virus (JEV) (e.g., GenBank® Database Accession No. U14163), West Nile virus (WNV) (e.g., GenBank® Database Accession No. DQ211652), tick-borne encephalitis virus (TBEV) (e.g., GenBank® Database Accession No. P14336) and any other flavivirus now known or later identified.

An amino acid residue of this invention can be a modified amino acid residue (nonlimiting examples are shown in Table 4 and/or can be an amino acid residue that is modified by post-translation modification (e.g., acetylation, amidation, formylation, hydroxylation, methylation, phosphorylation or sulfatation).

A “sample” or biological sample” of this invention can be any sample that can contain an antibody or polypeptide or antigen as described herein. Nonlimiting examples of a biological sample include blood, serum, plasma, saliva, urine, tears, semen, fecal matter, joint fluid, sputum, lavage fluid, cerebrospinal fluid, mucous, cells, tissue, etc.

It is contemplated that the polypeptides of this invention can be attached to, linked to and/or formed on a solid substrate. A solid substrate of this invention can be any solid surface to which the amino acid residues can attach in an orientation that allows for formation and/or folding of the polypeptide in a functional conformation, according to the methods described herein. In some embodiments, the solid substrate can be, but is not limited to a plate, resin, dish, slide, well, etc., as would be commonly used in an immunoassay or any other type of assay or reaction.

In further embodiments, the present invention provides the polypeptides of this invention immobilized on a solid support (e.g., beads, plates, slides or wells formed from materials such as latex or polystyrene). Examples of such solid supports include plastics such as polycarbonate, complex carbohydrates such as agarose and sepharose, acrylic resins and such as polyacrylamide and latex beads. Techniques for coupling proteins to such solid supports are well known in the art (Weir et al., Handbook of Experimental Immunology 4th Ed., Blackwell Scientific Publications, Oxford, England, Chapter 10 (1986)). Polypeptides can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

Various immunoassays can be used for screening to identify antibodies having specificity for the polypeptides of this invention. Numerous protocols for competitive binding or immunoradiometric assays are well known in the art. Such immunoassays typically involve the measurement of complex formation between an antigen and its specific antibody (e.g., antigen/antibody complex formation).

In certain embodiments, the polypeptides of this invention can be fused with a “carrier” protein or peptide to produce a fusion protein. Such fusion can be carried out, for example, by linking a nucleic acid of this invention in frame with a nucleic acid encoding a carrier protein or fragment thereof of this invention and expressing the linked nucleotide sequence to produce the fusion protein. For example, the carrier protein or peptide can be fused to a polypeptide and/or fragment of this invention to increase the stability thereof (e.g., decrease the turnover rate) in the cell and/or subject. Exemplary carrier proteins include, but are not limited to, glutathione-S-transferase or maltose-binding protein. The carrier protein or peptide can alternatively be a reporter protein. For example, the fusion protein can comprise a polypeptide and/or fragment of this invention and a reporter protein or peptide (e.g., green fluorescence protein (GFP),β-glucoronidase,β-galactosidase, luciferase, and the like) for easy detection of transformed cells and transgene expression. Any suitable carrier protein and/or nucleic acid encoding the carrier protein, as is well known in the art can be used to produce a fusion protein of this invention.

A variety of protocols for detecting the presence of and/or measuring the amount of specific antibodies in a sample, using the polypeptides of this invention are known in the art. Examples of such protocols include, but are not limited to, enzyme immunoassays (EIA), agglutination assays, immunoblots (Western blot; dot/slot blot, etc.), radioimmunoassays (RIA), immunodiffusion assays, chemiluminescence assays, antibody library screens, expression arrays, enzyme-linked immunosorbent assays (ELISA), radioimmunoassays (RIA), immunoprecipitation, Western blotting, competitive binding assays, immunofluorescence, immunohistochemical staining precipitation/flocculation assays and fluorescence-activated cell sorting (FACS). These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

In some embodiments of this invention, the solid substrate can be any type of carrier that has a surface to which amino acid residues and/or polypeptides can attach in an orientation that allows for epitope formation according to the methods described herein. In some embodiments, the solid substrate can be a microparticle or nanoparticle.

Exemplary types of nanoparticles of this invention include but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales.

Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art.

As used herein, the terms “nanoparticle” and “nanosphere” describe a polymeric particle or sphere in the nanometer size range. The term microparticle” or “microsphere” as used herein describes a particle or sphere in the micrometer size range. Both types of particles or spheres can be used as carriers of this invention.

A nanoparticle or nanosphere of this invention can have a diameter of 100 nm or less (e.g., in a range from about 1 nm to about 100 nm). In some embodiments, a particle with dimensions more than 100 nm can still be called a nanoparticle. Thus, an upper range for nanoparticles can be about 500 nm. A microparticle or microsphere of this invention can have a diameter of about 0.5 micrometers to about 100 micrometers.

In some embodiments, a particle of this invention can comprise a polymer that can be PLGA-based, PLA-based, and/or polysaccharide-based (dextran, cyclodextrin, chitosan, heparin etc.); a dendrimer; a hydrogel; a lipid base; a lipid hybrid base; a liposome; a micelle; an inorganic base such as, e.g., superparamagnetic iron oxide, metal, platin, calcium phosphate; a quantum dot; a carbon base, such as, e.g., a fullerene, a carbon nanotube; and a protein-based complex with nanoscales.

In some embodiments of this invention, the solid substrate can be a nanocapsule. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

In still further embodiments of the invention, the present invention provides peptide mimitopes (see, Meloen et al. (2000) J. Mol. Recognit. 13, 352-359) that mimic the individual and conformational epitopes of the polypeptides of the invention. Mimitopes may be identified using any technique known in the art, such as by surface stimulation, random peptide libraries or phage display libraries, using an antibody or antibodies to the individual and conformational epitopes of the polypeptides of the invention.

The invention further provides a nucleic acid molecule (e.g., isolated nucleic acid) encoding a polypeptide or peptide of this invention. Also provided are vectors (e.g., plasmids, viral vectors, etc.) encoding the nucleic acid molecules of the invention.

Also provided are cells comprising the polypeptides, peptides, nucleic acid molecules, vectors, virus particles and/or VLPs of this invention.

The term “epitope” as used herein means a specific combination of amino acid residues that, when present in the proper conformation, provide a reactive site for an immune response, e.g., involving an antibody (e.g., B cell epitope) and/or T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715.

Similarly, conformational epitopes can be readily identified by determining spatial conformation of amino acids such as by, e.g., cryoelectron microscopy, x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes that are involved in stimulating the cellular arm of a subject's immune system are short peptides of about 8-25 amino acids. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenically active fragments can also be identified based on their ability to stimulate lymphocyte proliferation in response to stimulation by various fragments from the antigen of interest.

The present invention can be practiced for prophylactic, therapeutic and/or diagnostic purposes. In addition, the invention can be practiced to produce antibodies for any purpose, such as diagnostic or research purposes, or for passive immunization by transfer to another subject.

As used herein, the term “nucleic acid” and “nucleic acid molecule” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

A “recombinant” nucleic acid molecule, polynucleotide or nucleotide sequence is a synthetic molecule that is not found in nature and is produced by genetic engineering techniques.

A “recombinant” polypeptide is a synthetic molecule that is not found in nature and is produced from a recombinant nucleic acid molecule, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

“Antibody” as used herein refers to an intact immunoglobulin of any isotype, or a fragment thereof that can compete with the intact antibody for specific binding to the target antigen, and includes chimeric, humanized, caninized, equinized, felinized, fully human, fully canine, fully equine, fully feline, and bispecific antibodies. An intact antibody generally will comprise at least two full-length heavy chains and two full-length light chains, but in some instances may include fewer chains such as antibodies naturally occurring in camelids which may comprise only heavy chains. Antibodies according to the invention may be derived solely from a single source, or may be “chimeric,” that is, different portions of the antibody may be derived from two different antibodies. For example, the CDR regions may be derived from a rat or murine source, while the framework regions of the V region are derived from a different animal source, such as a human. The antibodies or binding fragments of the invention may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below.

“Light chain” as used herein includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains according to the invention include kappa chains and lambda chains.

“Heavy chain” includes a full-length heavy chain and fragments thereof having sufficient variable region sequence to confer binding specificity. A full-length heavy chain includes a variable region domain, V_{H}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the —COOH end. Heavy chains according to the invention may be of any isotype, including IgG (including IgG1, IgG2, IgG3 and IgG4 subtypes), IgA (including IgA₁ and IgA₂ subtypes), IgM and IgE.

“Immunologically functional fragment” (or simply “fragment”) of an immunoglobulin chain, as used herein, refers to a portion of an antibody light chain or heavy chain that lacks at least some of the amino acids present in a full-length chain but which is capable of binding specifically to an antigen. Such fragments are biologically active in that they bind specifically to the target antigen and can compete with intact antibodies for specific binding to a given epitope. In one aspect of the invention, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of intact antibodies. Immunologically functional immunoglobulin fragments of the invention include, but are not limited to, Fab, Fab′, F(ab′)₂, Fv, domain antibodies and single-chain antibodies, and may be derived from any mammalian source, including but not limited to human, mouse, rat, camelid or rabbit. It is contemplated further that a functional portion of the antibodies, for example, one or more CDRs, could be covalently bound to a second protein or to a small molecule to create a therapeutic agent directed to a particular target in the body, possessing bifunctional therapeutic properties, or having a prolonged serum half-life.

“Fab fragment” as used herein is comprised of one light chain and the CHI and variable regions of one heavy chain. The heavy chain of a Fab molecule cannot form a disulfide bond with another heavy chain molecule.

“Fc” region as used herein contains two heavy chain fragments comprising the C_{H1} and C_{H2} domains of an antibody. The two heavy chain fragments are held together by two or more disulfide bonds and by hydrophobic interactions of the CH3 domains.

“Fab′ fragment” contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form a F(ab′)₂ molecule.

“F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference.

“Domain antibody” as used herein is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

“Bivalent antibody” as used herein comprises two antigen binding sites. In some instances, the two binding sites have the same antigen specificities. However, bivalent antibodies may be bispecific (see below).

“Multispecific antibody” as used herein is one that targets more than one antigen or epitope.

“Bispecific,” “dual-specific” or “bifunctional” antibody as used herein is a hybrid antibody having two different antigen binding sites. Bispecific antibodies are a species of multispecific antibody and may be produced by a variety of methods including, but not limited to, fusion of hybridomas or linking of Fab′ fragments. See, e.g., Songsivilai & Lachmann (1990) Clin. Exp. Immunol. 79:315-321; Kostelny et al. (1992) J. Immunol. 148:1547-1553. The two binding sites of a bispecific antibody will bind to two different epitopes, which may reside on the same or different protein targets.

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of flavivirus infection.

“Effective amount” as used herein refers to an amount of a vector, nucleic acid, epitope, polypeptide, cell, particle, VLP, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination” and “immunization” are well-understood in the art, and are used interchangeably herein. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing Zika virus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating flavivirus infection in a subject, whether against one or multiple strains, genotypes or serotypes of a flavivirus such as dengue virus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” (Herscowitz, Immunophysiology: Cell Function and Cellular Interactions in Antibody Formation, in IMMUNOLOGY: BASIC PROCESSES 117 (Joseph A. Bellanti ed., 1985)). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “subject” of the invention includes any animal susceptible to flavivirus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. A subject of the invention can be a subject known or believed to be at risk of infection by flavivirus. Alternatively, a subject according to the invention can also include a subject not previously known or suspected to be infected by flavivirus or in need of treatment for flavivirus infection. In particular embodiments, the subject is a pregnant female. A pregnant female subject can be tested according to the methods of this invention for Zika virus infection and/or prior exposure to Zika virus. The pregnant female subject can be monitored over time before, during and/or after pregnancy. The fetus and/or neonate can be tested for Zika virus infection and/or exposure to Zika virus and/or monitored over time.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age.

Subjects may be treated for any purpose, such as for eliciting a protective immune response, for eliciting a neutralizing response, and/or for eliciting the production of antibodies in that subject, which antibodies can be collected and used for other purposes such as research or diagnostic purposes or for administering to other subjects to produce passive immunity therein, etc.

In embodiments of the invention, the subject has maternal antibodies to a flavivirus of this invention.

A “subject in need” of the methods of the invention can be a subject known to be, or suspected of being, infected with, or at risk of being infected with, a flavivirus of this invention. A subject of this invention can include a woman of child-bearing age, a pregnant woman, a sex partner, a fetus, etc.

Pharmaceutical formulations (e.g., immunogenic formulation) comprising the polypeptides, and/or other compositions of the invention and a pharmaceutically acceptable carrier are also provided, and can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of the invention is typically admixed with inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of the invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

In some embodiments, the compositions of the invention can further comprise one or more than one adjuvant. The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al., Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art. In embodiments of the invention, the adjuvant comprises an alphavirus adjuvant as described, for example in U.S. Pat. No. 7,862,829.

An adjuvant for use with the present invention, such as, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science and Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition of this invention can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The pharmaceutical compositions that are provided can be administered for prophylactic and/or therapeutic treatments. An “effective amount” refers generally to an amount that is a sufficient, but non-toxic, amount of the active ingredient to achieve the desired effect, which is a reduction or elimination in the severity and/or frequency of symptoms and/or improvement or remediation of damage. A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom.

In general, toxicity and therapeutic efficacy of the immunogenic composition and/or vaccine of this invention can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for subjects for treatment. The dosage of the active ingredient typically lines within a range of circulating concentrations that include the ED₅₀ with little or no toxicity. The dosage can vary within this range depending upon the dosage form employed and the route of administration utilized.

The effective amount of a pharmaceutical composition of this invention to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the anti-composition is being used, the route of administration, and the size (body weight, body surface or organ size) and/or condition (the age and general health) of the subject. A clinician may titer the dosage and modify the route of administration to obtain the optimal therapeutic effect. Typical dosages can range from about 10 or 100 ug/Kg, or 500 or 1 mg/Kg, up to about 50 or 100 mg/Kg subject body weight, or more.

In some embodiments of the invention, the dosage of a protein (e.g., a composition comprising a polypeptide of this invention or a polypeptide linked to a carrier such as a nanoparticle) can be in a range of about 10° to about 10⁴ micrograms, +/−adjuvant.

The dosing frequency will depend upon the pharmacokinetic parameters of the composition being administered. For example, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

Kits that include a polypeptide and/or other composition as described herein are also provided. Some kits include such a polypeptide or composition and may also include instructions for use of the polypeptide or composition in the various methods disclosed above. The polypeptide or composition can be in various forms, including, for instance, as part of a solution or as a solid (e.g., lyophilized powder). The instructions may include a description of how to prepare (e.g., dissolve or resuspend) the polypeptide or composition in an appropriate fluid and/or how to administer the polypeptide or composition for the treatment and/or prevention of the disorders and/or conditions described.

The kits may also include various other components, such as buffers, salts, complexing metal ions and other agents described above in the section on pharmaceutical compositions. These components may be included with the polypeptide or composition and/or may be in separate containers. The kits may also include other therapeutic agents for administration with the polypeptide and/or composition and/or vaccine. Examples of such agents include, but are not limited to, agents to treat the disorders or conditions as described herein.

The present invention further provides a kit comprising one or more compositions of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other reagent and/or solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

The present invention is more particularly described in the following examples, which are intended as illustrative only since numerous modifications and variations therein will be apparent to those skilled in the art.

### EXAMPLES

### Example 1. Development of Envelope Protein Antigens to Serologically Differentiate Zika from Dengue Virus Infection

Zika virus (ZIKV) is an emerging flavivirus that can cause birth defects and neurologic complication. Molecular tests are effective in diagnosing acute ZIKV infection, although the majority of infections produce no symptoms at all or present after the narrow window in which molecular diagnostics are dependable. Serology is a reliable method for detecting infections after the viremic period; however, most serological assays have limited specificity due to cross-reactive antibodies elicited by flavivirus infections. Since ZIKV and dengue virus (DENV) widely co-circulate, distinguishing Zika from dengue virus infection is particularly important for diagnosing individual cases or surveillance to coordinate public health response. Flaviviruses also elicit type-specific antibodies directed to non-cross-reactive epitopes of the infecting virus; such epitopes are attractive targets for designing antigens to develop serologic tests with greater specificity.

Guided by comparative epitope modeling of ZIKV envelope protein, we hypothesized that divergent regions within the putative epitopes may be targeted by type-specific antibodies. We designed two recombinant antigens displaying unique antigenic regions on domain I (Z-EDI) and domain III (Z-EDIII) of ZIKV envelope protein. Both Z-EDI and Z-EDIII antigens consistently detected ZIKV-specific IgG in ZIKV- but not DENV-immune sera in later convalescence (>12 weeks post-infection). In contrast, neither Z-EDI nor Z-EDIII differentiate ZIKV from DENV in early convalescence (2-12 weeks post-infection) due to cross-reactive IgG antibodies. Our study provides a framework for further optimization of Z-EDI- and Z-EDIII-based assays for laboratory diagnosis of ZIKV infection.

Zika virus (ZIKV) is an enveloped, positive-sense, single-stranded RNA virus in the flavivirus genus, which includes other medically important viruses such as dengue virus (DENV), West Nile virus, and yellow fever virus. ZIKV infection has become a major global health concern as it can disseminate rapidly in naïve populations, lead to neurologic sequelae such as a Guillain-Barré-like syndrome in otherwise healthy individuals, and cause developmental abnormalities including ocular damage, microcephaly, and fetal death when infection occurs during pregnancy. People at risk of DENV infection are also at risk of ZIKV infection as both are transmitted by Aedes aegypti mosquitoes. ZIKV also has the unusual ability among flavivirus to be transmitted through sexual contact and from mother to fetus during pregnancy.

Accurate diagnosis is critical to many aspects of the public health response to ZIKV epidemic, but can be complicated by multiple factors. Clinically, it is impossible to discern between myriad causes of acute fever and/or rash. Molecular tests are useful for detecting symptomatic flavivirus infections during the brief period immediately following infection. However, most individuals with ZIKV infection never seek medical attention because they are asymptomatic or experience only a mild, self-limited illness. Beyond this acute period, serological tests are necessary to detect ZIKV infections and to support public health efforts such as prenatal evaluation and management, risk reduction counseling, and surveillance and outbreak investigations.

Unfortunately, most serological tests lack specificity due to cross-reactive antibodies elicited by flavivirus infections. Neutralization assays, which are more specific but less widely available due to their resource-intensive nature, may or may not clarify IgM results that suggest ZIKV or DENV infection, leaving many waiting weeks for a diagnosis or receiving the ambiguous designation of “recent flavivirus infection.” Patient serum collected 5 or more days after onset of symptoms contains a complex mixture of antibody populations against the viral envelope (E) protein directed to epitopes that are unique to the infecting virus as well as epitopes that are conserved among flaviviruses. Consequently, assays that employ the whole virus or E as antigen do not reliably distinguish infections caused by ZIKV from DENV. Recombinant ZIKV antigens containing epitopes recognized by type-specific but not cross-reactive antibody are needed to develop serological diagnostic assays with greater specificity for ZIKV infection.

The surface of the ZIKV virion is decorated by 180 copies of E with icosahedral symmetry. Each E protein monomer is composed of an amino terminal ectodomain (E80; 1-403 aa), two amphipathic α-helices and two carboxy terminal membrane-spanning α-helices. The surface-exposed E80 comprises three distinct domains (EDI, EDII and EDIII) with EDI in the center. The domains EDI (1-49; 136-195; 286-302 aa) and EDII are non-contiguous in sequence and are connected by a flexible hinge region (EDI/II hinge), whereas EDIII (303-403 aa) is a continuous domain extending from EDI.

Here we present the design, production and evaluation of ZIKV EDI and EDIII antigens (hereafter referred to as Z-EDI and Z-EDIII) for serological diagnosis of Zika virus (ZIKV) using well-characterized early and late convalescent immune sera from individuals following, DENV, or both.

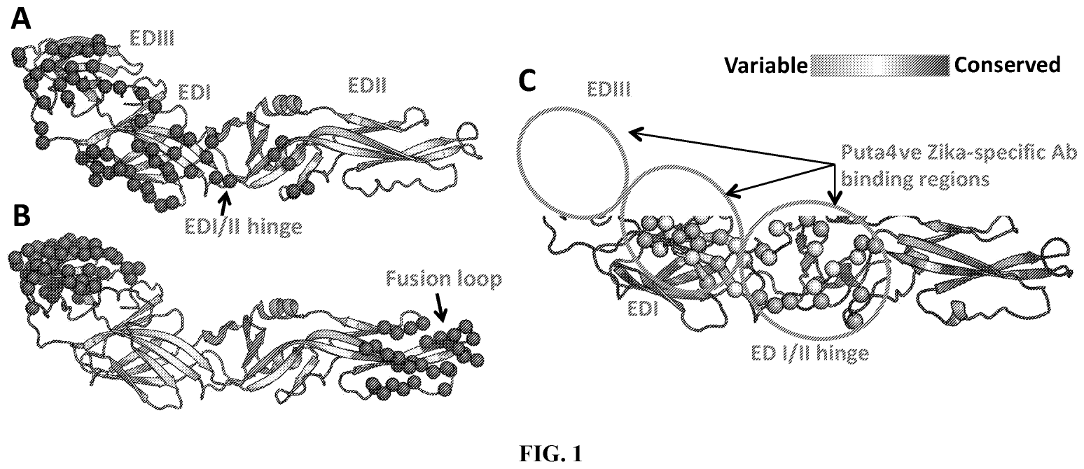

Computational prediction of ZIKV-specific antibody binding regions. ZIKV E protein shares 55-58% sequence identity with DENV E proteins and contains highly conserved epitopes that are responsible for extensive cross-reactivity with polyclonal serum antibodies. However, people infected with ZIKV develop antibodies that neutralize ZIKV but not DENV demonstrating the presence of epitopes that are unique to ZIKV. To identify E protein antigenic regions that may be targets for ZIKV-specific antibodies, we generated and compared surface maps of known DENV antibody epitopes and a map of surface amino acid conservation between different flaviviruses including ZIKV and the 4 DENV serotypes (FIG. 1, Panel A and Panel B). Surface amino acid sequence conservation analysis is successfully used to identify conserved and variable regions in protein. Our rationale is that such conservation analysis, when combined with the knowledge of conformational epitopes of E protein of DENV, may prove useful to accurately predict ZIKV-specific antigenic regions.

To perform comparative epitope mapping of E protein, we superimposed experimentally determined structures for type-specific and cross-reactive antibody/E protein complexes to a reference E structure. Analyzing the residues at the interface between E protein and antibody complex showed that there are two possible cross-reactive antibody binding sites on the surface of E protein: one site is at the tip of EDII, which contains the fusion-loop and the other site is located on the EDIII surface formed by β-strands A, B and E and G (FIG. 1, Panel A). Next, we used the Consurf algorithm to obtain a conservation score for each amino-acid position across 8 different E proteins from clinically relevant flaviviruses (FIG. 1, Panel C). Projecting the Consurf conservation score onto the molecular surface of the ZIKV E structure showed that most of the solvent exposed outer surface is variable between flaviviruses, whereas the surface adjacent to the stem region, the transmembrane helices and the regions contributing to inter-molecular assembly are largely conserved. The correlation between cross-reactive epitopes and the conserved regions, and between virus-specific epitopes and variable regions were evident across the maps compared. Accordingly, we identified three regions that may be recognized by ZIKV type-specific antibodies: a region around the solvent exposed “glycosylation loop” on EDI and the edge of the EDI, a region on the outer surface of the flexible hinge region formed between EDI and EDII, and a region on the “lateral ridge” of EDIII (FIG. 1, Panel C).

Expression of ZIKV Recombinant Antigens.

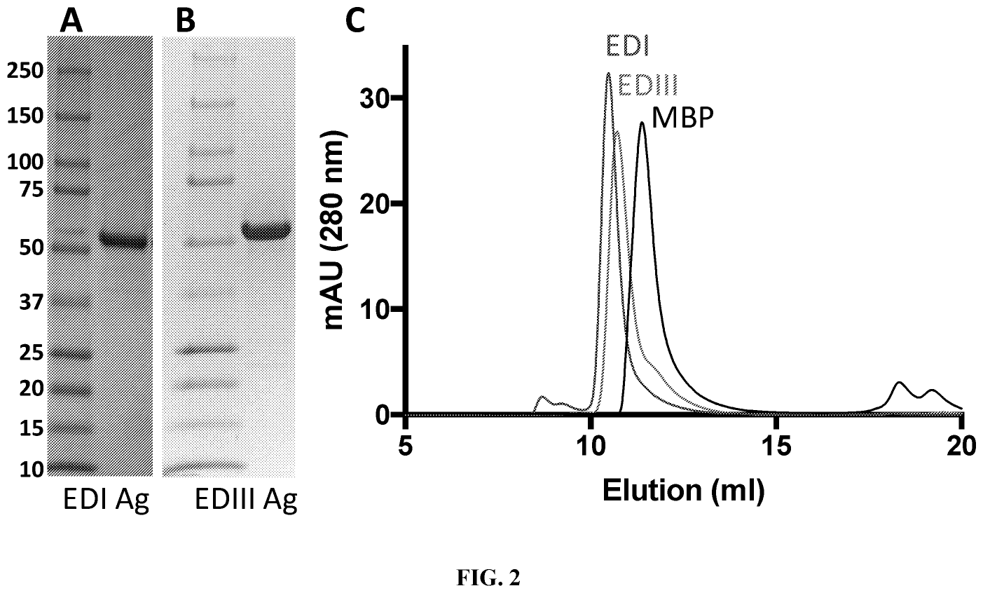

Following our prediction that epitopes recognized by ZIKV type-specific antibodies are mainly located on EDI and EDIII, we designed two constructs of Z-EDI and Z-EDIII fused to maltose binding protein (MBP) for periplasmic expression in E. coli. Soluble recombinant Z-EDI and Z-EDIII were readily purified by amylose affinity chromatography, yielding ˜3 mg of purified protein from one liter of bacterial culture (FIG. 2, Panel A and Panel B). Size exclusion chromatography analysis showed that the recombinant antigens behaved as monomeric proteins in solution (FIG. 2, Panel C) and the Ellman assay confirmed the presence of intact intramolecular disulfide bond in Z-EDI and Z-EDIII antigens. Moreover, Z-EDIII was able to bind to the mouse monoclonal antibodies ZV-2, ZV-48 and ZV-67 that recognize conformational epitope. We also expressed the entire ectodomain of ZIKV E protein (Z-E80) to use as a reference antigen to evaluate the performance of Z-EDI and Z-EDIII.

Immune Sera from People Exposed to DENY and ZIKV.

To evaluate recombinant antigens for serological detection of ZIKV infection, we assembled panels of 22 late convalescent samples (collected >12 weeks after infection) and 25 early convalescent samples (collected between 2 weeks and 12 weeks after infection) from individuals who were exposed to ZIKV, DENV or both through travel or residence in endemic areas (Tables 1 and 2). We categorized the serostatus of each sample in the panels as primary flavivirus-immune specimens (neutralizing activity to only one serotype of DENV or ZIKV), secondary flavivirus-immune specimens (neutralizing activity to more than one serotype of DENV or both ZIKV and DENV) and naïve specimens (no neutralizing activity to DENV or ZIKV).

Evaluation of ZIKV E80, EDI and EDIII Antigens for Serological Detection of Remote (>12 Weeks) Infections.

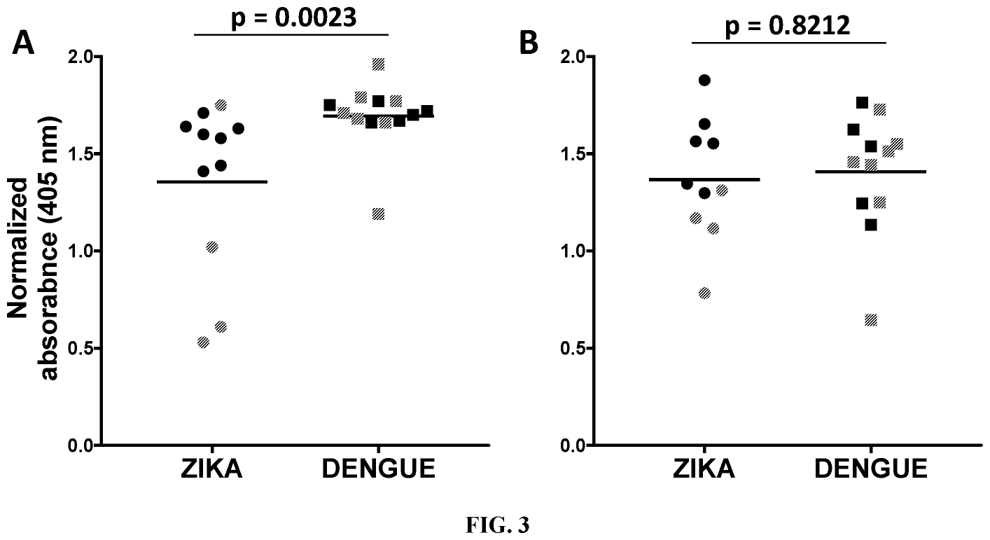

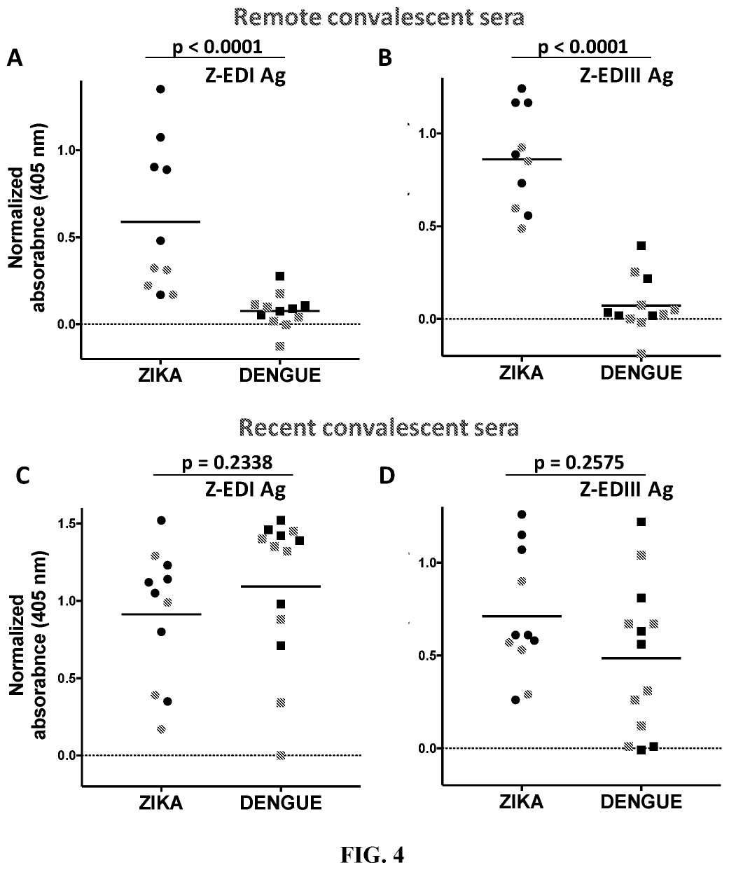

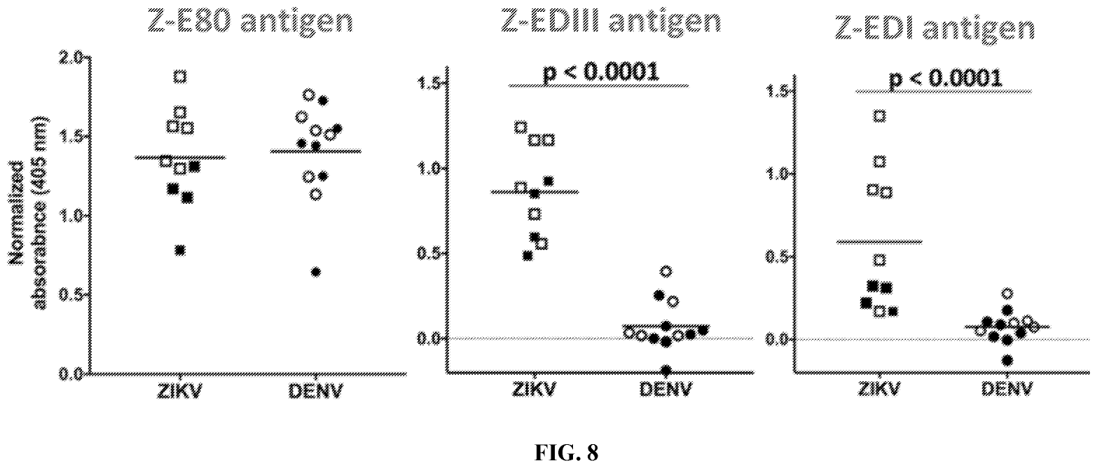

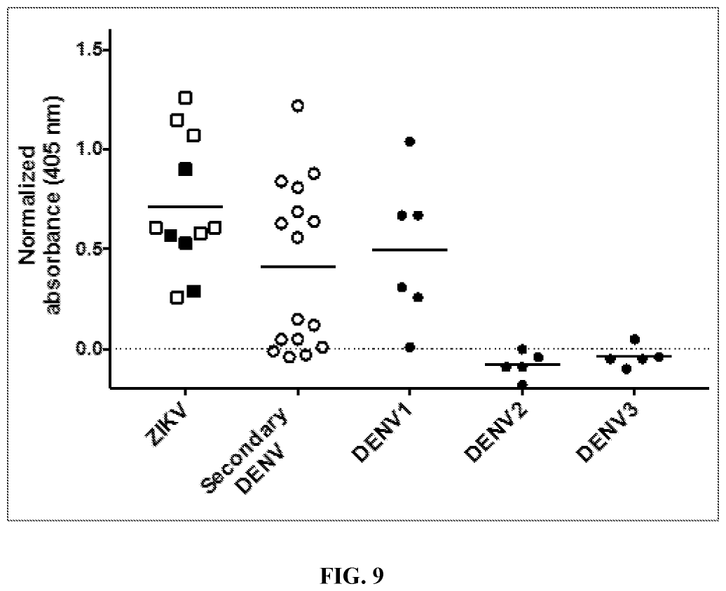

Although ZIKV-immune sera reacted strongly with ZIKV E80, immune sera from individuals infected with DENV consistently showed high levels of cross-reactivity with recombinant ZIKV E80 antigen in a standard IgG ELISA (FIGS. 3, 8). Using an anti-MBP monoclonal antibody to capture MBP-fusion proteins, we developed a sandwich ELISA to measure serum IgG levels to Z-EDI and Z-EDIII (FIG. 4, Panel A and Panel B; FIG. 9). At late convalescence, immune sera from Zika cases recognized Z-EDIII and Z-EDI antigens significantly better than immune sera from DENV cases (p<0.0001 Mann-Whitney test). Consequently, Z-EDI and Z-EDIII antigens may be useful for specific detection of remote (>12 weeks) ZIKV infections in areas with endemic DENV transmission.

Evaluation of ZIKV E80, EDI and EDIII Antigens for Serological Detection of Recent (Between 2 and 12 Weeks) Infections.

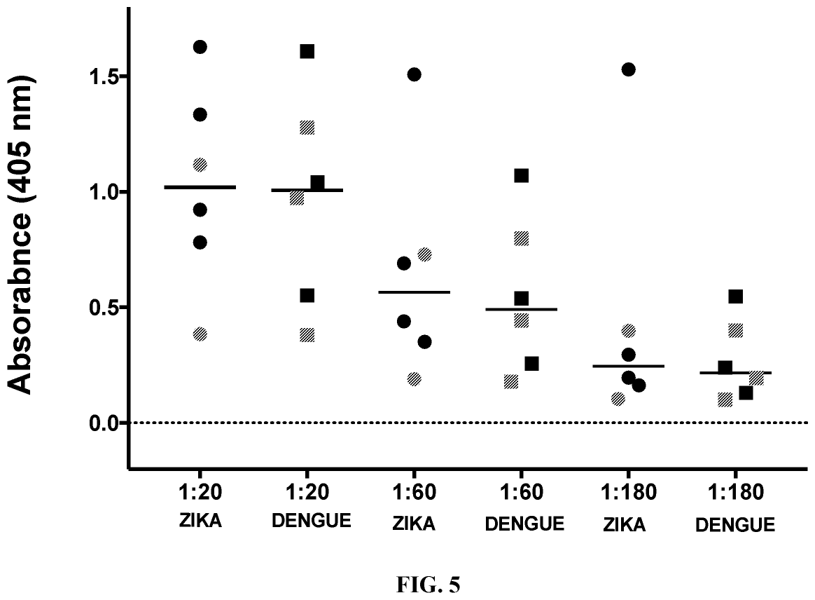

At early convalescence, immune sera collected from ZIKV infected individuals had high levels of IgG that bound to Z-E80, Z-EDI and Z-EDIII. Remarkably, even the DENV-immune sera collected during the early convalescent phase reacted strongly with Z-EDI and Z-EDIII antigens. As our initial IgG assays were performed using a 1:20 dilution of serum, we further diluted the early convalescent samples in an attempt to improve specificity. Dilution of early-convalescent-phase serum to dilutions of up to 1:180 was not adequate to improve the specificity of Z-EDI and Z-EDIII against secondary DENV- or DENV1-immune sera (FIG. 5). Taken together, IgG cross-reactivity with Z-EDI and Z-EDIII antigens in DENV-immune sera was pronounced in the early convalescent phase (<12 weeks) but not at the late convalescent (>12 weeks) phase.

As ZIKV is emerging in areas with intense DENV transmission and, more recently, testing of DENV vaccines, there is urgent need for simple serological assays to distinguish ZIKV from DENV infections. Our comparative analysis of surface amino acid conservation among flavivirus E proteins and homology epitope mapping pointed to three regions on ZIKV E protein as potential targets of ZIKV type-specific antibodies. Here, we evaluated the utility of recombinant Z-EDI and Z-EDIII antigens, which display two of the three predicted ZIKV-specific antigenic regions. Our results demonstrate that Z-EDIII and, to a lesser extent, Z-EDI are strong candidate antigens for serological tests that differentiate ZIKV from DENV infections when samples are collected >12 weeks after infection. The recombinant antigens performed equally well for both primary and secondary infection samples, indicating that specificity was not reduced by high levels of cross-reactive antibodies characteristic of secondary flavivirus infection.

Dengue virus induced antibody responses are mainly targeted against the envelope (E) protein. Many non-neutralizing antibodies are cross-reactive between the 4 different DENV serotypes (DENV-1-4) and recognize specific epitopes on E that do not attribute to the protection against DENV infections. Highly potent neutralizing antibodies are often targeted against epitopes that require higher order quaternary protein structures that are assembled and displayed on intact virions only. Between serotypes, the neutralizing epitopes differ in structure, complexity and location. These serotype specific neutralizing antibodies render protection against subsequent virus infections of the same serotype.

In contrast to late convalescence, we observed a high level of cross-reactivity in early dengue convalescent samples (2-12 weeks after infection). At early convalescence, individuals exposed to either DENV or ZIKV infections had similar levels of antibodies that bound to Z-EDI and Z-EDIII. We hypothesize that a distinct population of transient, flavivirus cross-reactive IgG antibodies that recognize conserved regions on Z-EDI and Z-EDIII are responsible for this cross-reactivity, which could lead to poor specificity in a diagnostic assay. In addition, individuals with prior-exposure to DENV1 infection have been recently reported to have high levels of cross-reactive antibodies to Z-EDIII. Over time, the cross-reactive antibodies, particularly the cross-neutralizing antibodies, decline, whereas type-specific responses are more stable and may even increase. While the cellular mechanisms responsible for the differential decline of cross-reactive and type-specific serum antibodies are not known, this phenomenon may be responsible for patterns of cross-reactivity with Z-EDI and Z-EDIII. One possible explanation is that many of the cross-reactive antibodies are derived from plasmablasts or extra-follicular B cells that are not maintained as long-lived plasma cells or memory B-cells.

Development of serological tests for diagnosing ZIKV infection in the context of prior flavivirus infection is a challenging and complex problem that remains a major unmet need. To date, there are only three serologic assays for ZIKV approved by the United States Food and Drug Administration under an emergency use authorization (fda.gov/MedicalDevices/Safety/EmergencySituations/ucm161496.htm#zika), and a few other commercial tests are available in countries outside the USA or for research purposes. These assays use either NS1, recombinant E, or other unspecified ZIKV antigen. The Centers for Disease Control and Prevention MAC (IgM) ELISA exhibits well-publicized limitations including false negatives, false positive results due to cross-reactive antibody from DENV infection, and persistence of ZIKV IgM beyond the previously presumed 12-week window. Our findings of cross-reactive IgG binding in early convalescence indicate that this time period will be the most challenging to optimize specificity of assays (thus, there is roughly a 10-week period (week 2-12) following infection when current and next generation serodiagnostics may remain ambiguous).

Additional issues preclude optimal implementation of many currently available serologic assays. In general, the serum panels used to evaluate these assays come from remnant clinical specimens or archived serum not collected systematically and specifically for analysis of clinical performance in diagnosing individuals with multiple flavivirus exposures. Sera from individuals with single flavivirus infection history and residing in regions not endemic for flavivirus infection are not representative of the populations for whom improved diagnostics is most critical—namely, those residing in the tropics, where individuals experience multiple and frequent flavivirus exposures. We are involved with ongoing studies designed to address this shortcoming. Sensitivity in different IgM assays can be less than 80%, particularly outside of the range of 6-60 days, when IgM assays perform best. Finally, not only have false positive ZIKV tests been reported due to current or previous DENV infection, DENV tests may also be positive following confirmed ZIKV infections. The cumulative experience with ZIKV serodiagnosis to date clearly indicates that novel approaches will be required.

Diversity in infecting strains of ZIKV may elicit antibodies that target different epitopes or different permutations of the same antigenic region of E protein. While we only evaluated a single construct for each of the Z-EDI and Z-EDIII antigens, we believe these antigens are likely to be representative of the vast majority of ZIKV strains in circulation. In fact, E protein amino acid sequences from ZIKV isolates from several different times and places vary by only <1%, and both African and Asian lineage strains perform similarly in binding and neutralization assays, suggesting that ZIKV exists as a single serotype. While the present work provides the platform for incorporating Z-EDI and Z-EDIII into a suitable antigen-antibody binding assay for the purpose of surveillance and risk reduction counseling, further modification of Z-EDI and Z-EDIII are necessary to fully utilize these antigens in the early convalescence phase of ZIKV infection. Cross-reactive antibodies may be depleted using recombinant dengue antigens, but depletion techniques are tedious and time consuming. Introducing amino-acid variation through protein engineering is an attractive strategy to eliminate cross-reactive antibody binding sites, while preserving unique epitopes within Z-EDIII and Z-EDI antigens. The high signal we gained for Z-EDIII with simple ELISA format is encouraging, although a combination of Z-EDI and Z-EDIII as well as fusion of antigens to protein scaffolds may also be tested for improving the sensitivity of assay. Finally, one interesting observation within our data is that some individuals are strongly IgG seropositive for one of the two recombinant antigens we tested, raising the possibility that a multiplex platform employing a panel of antigens may improve sensitivity.

A recent report provides proof of this principle (although 36% of ZIKV cases resulted in a false positive signal for anti-DENV NS1 IgM). This approach also has the advantage of designing expanded antigen panels to detect antibody specific for additional pathogens that cause similar clinical presentations as DENV and ZIKV.

In conclusion, we have demonstrated that Z-EDI and Z-EDIII contain important epitopes that can be used to resolve serodiagnostic problems facing ZIKV-endemic and non-endemic areas. Ultimately, this work can lead to development of crucial diagnostic tools, including ones amenable to field use in resource-limited settings. In the process, much can be learned about the epitopes targeted by durable type-specific and cross-reactive human antibodies generated upon ZIKV exposure, which is important for the design of highly efficacious DENV and ZIKV vaccines.

Human Subjects and Clinical Specimens.

Sera were collected from North Carolina residents or visitors with possible or confirmed DENV or ZIKV infection based on travel to or prior residence in endemic areas and self-reported symptoms. All human specimens were de-identified. All UNC donations were collected in compliance with the Institutional Review Board of the University of North Carolina at Chapel Hill (protocol 08-0895). An additional set of immune sera were obtained from cohort studies in Nicaragua, Colombia, Brazil and Sri Lanka.

Samples from Nicaragua.

Five children who were RT-PCR-positive for ZIKV who experienced onset of signs and symptoms of Zika, from the Nicaraguan Pediatric Dengue Cohort Study (PDCS) were included. The PDCS is a community-based prospective study of children 2 to 14 years of age that has been ongoing since August 2004 in Managua, Nicaragua. Participants present at the first sign of illness to the Health Center Socrates Flores Vivas and are followed daily during the acute phase of illness. Acute and convalescent (˜14-21 days after onset of symptoms) blood samples are drawn for dengue, chikungunya and Zika diagnostic testing from patients meeting the case definition for dengue or Zika (starting in January 2016) or presenting with undifferentiated febrile illness. All Zika suspected cases were confirmed by RT-PCR in serum and/or urine using triplex assays that simultaneously screen for DENV and CHIKV infections (ZCD assay, CDC Trioplex) or in some cases the CDC ZIKV monoplex assay in parallel with a DENV-CHIKV multiplex assay. The PDCS was approved by the Institutional Review Boards of the Nicaraguan Ministry of Health and the University of California, Berkeley. Parents or legal guardians of all subjects provided written informed consent, and subjects ≥6 years old provided assent.

Samples from Colombia.

Sera samples were also collected in Sincelejo, Colombia as part of a field investigation of the Zika outbreak and arboviruses surveillance program conducted by the University of Sucre. All participants signed an informed consent prior the blood collection, as described in the University of Sucre Bioethics Committee approved protocol. Samples were collected during the convalescence phase (3 months after symptoms onset) from participants that reported Zika-related symptoms; all sera separation procedures were performed under BSL2 cabinets to ensure the quality of the samples.

Samples from Brazil.

A cohort of pregnant women with confirmed or suspected Zika virus infection during pregnancy in Vitoria, Brazil, where women with Zika-like illness were enrolled in a clinical study to follow Zika and other related viruses by testing, viremia, and clinical outcome of the mother-infant pair, under an approved protocol from the national and local IRB.

Samples from Sri Lanka.

Sri Lankan serum samples were collected in the convalescent phase from patients with confirmed dengue virus infection. Acute infection was confirmed by detection of virus (PCR+) and/or DENV-specific IgM and IgG in the serum. Samples were collected 2-12 weeks after infection as previously described. The IRBs of both LH and the Medical Faculty, University of Colombo (serving as NIH approved IRB for Genetech) approved all protocols described in this study.

Sera were heat-inactivated at 56° C. for 30 min. Serostatus of specimens were categorized into primary or secondary infection using neutralization assay as previously described. Five-fold diluted sera were mixed with 50-100 focus-forming units of virus per well in Dulbecco's modified Eagle medium supplemented with 2% FBS. Virus-antibody mixtures were incubated for 1 hour at 37° C. and then transferred to a confluent monolayer of Vero cells and then overlaid with media containing 1% methylcellulose. Infected cell foci were detected at 48 hr after infection, following fixation with 4% paraformaldehyde and incubation with 500 ng/ml of flavivirus cross-reactive mouse monoclonal antibody E60 for 2 hr at room temperature. After incubation for 1 hr with a 1:5,000 dilution of horseradish peroxidase (HRP)-conjugated goat anti-mouse IgG (Sigma), foci were detected by addition of TrueBlue substrate (KPL). Foci were analyzed with a CTL Immunospot instrument. IC50 values were calculated using the sigmoidal dose response (variable slope) equation in Prism 7 (Graphpad Software). Reported values were required to have an R²>0.75, a hill slope >0.5, and an IC50 within the range of the assay. Characterization of early sera that had neutralizing antibodies to one DENV serotype or to ZIKV with minimal (4× less than highest titer) cross-neutralizing antibodies were defined as primary flavivirus infections. Sera that had high levels of neutralizing antibodies to two or more flaviviruses were defined as repeat (secondary) flavivirus infections. Most secondary infection sera were from subjects who had resided in endemic countries.

Protein Production.

A codon-optimized gene encoding for Z-EDI or Z-EDIII from ZIKV strain H/PF/2013 was cloned into pET PPL His6 MBP expression vector (2K-T) using a ligation independent cloning method. The 2K-T plasmid was a gift from Scott Gradia (Addgene plasmid #37183). MBP fused to Z-EDI or Z-EDIII were expressed in BL21 (DE3)pLysS and purified using amylose affinity resin. ZIKV (aa 1-404) E80 antigen was expressed in Expi293 transient expression system and purified by Ni-NTA affinity resin as previously described.

IgG ELISA.

Human serum IgG binding was measured using ELISA assays as previously described. Recombinant ZIKV E80 antigen (500 ng/well) was coated on to the plate, blocked with 3% milk, and incubated with human serum at indicated dilution at 37° C. for 1 hour. Z-EDIII and Z-EDI sandwich ELISAs were the same as above, except that the antigens (200 ng/well) were captured by murine anti-maltose binding protein monoclonal antibody (New England Biolabs Inc). Bound IgG was detected with an alkaline phosphatase-conjugated anti-human secondary antibody by incubation with p-nitrophenyl phosphate substrate (Sigma), and absorbance at 405 nm was measured on an Epoch plate reader (BioTek). Mean binding signal for each serum was calculated from duplicates by subtracting the mean absorbance of background signal obtained from positive serum with no antigen (for ZIKV E80) or MBP (for Z-EDIII and Z-EDI). Statistical analysis was performed using the Mann-Whitney U test in Prism 7.0b for nonparametric comparison of recombinant antigen reactivity between sera from ZIKV and DENV patients.

Molecular Modeling and Structural Analysis.

For amino acid conservation analysis by ConSurf, eight flavivirus E protein sequences (ZIKV, four serotypes of DENV, Saint Louis encephalitis virus, Japanese encephalitis virus and yellow fever virus) were used. The ConSurf algorithm assigns relative conservation scores to each residue and normalizes the score such that the average is zero, and negative and positive deviations denote the degrees of conservation and variation, respectively. The relative conservation score is then converted to a value between 1 and 9 (1 for most variable (cyan), 5 for average (white), up to 9 for most conserved (purple)) to generate a heat map that is used to color the molecular surface of ZIKV E protein structure.

For type-specific epitope mapping, structures of monoclonal antibody complexes with E or E fragment (PDB IDs: 4UIF, 5A1Z, 4UIH, 3IYW, 4C2I, 3J05, 3J6U, 3UAJ, 3UC0, and 1ZTX) were aligned to reference E protein by use of PyMol software (The PyMOL Molecular Graphics System, Version 1.8 Schrödinger, LLC). For cross-reactive epitope mapping, antibody structure complex with E or E fragment (PDB IDs: 4UT9, 4UT6, 4UTA, 3150, 2R29, 3UZQ, 4FFY, SAAM, 4L5F, 4BZ2, 4AL8, 3UYP, 3UZE, and 3UZV) were aligned to reference E protein by use of Pymol software. Contact residues in the E protein-antibody interface were then identified by a 5.0 Å cut-off distance between any atoms in E to any atom in the antibody. All molecular figures were drawn with PyMOL.

### Example 2. Longitudinal Analysis of Ab Response to Z-EDIII

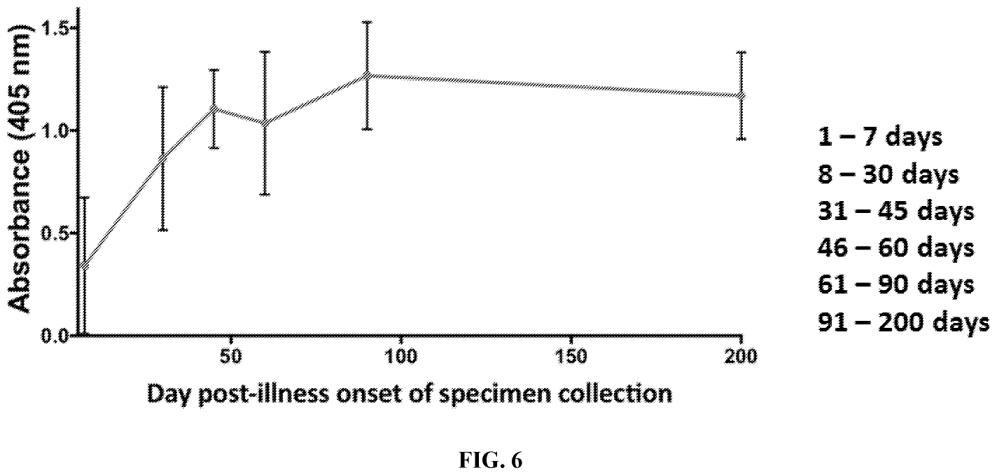

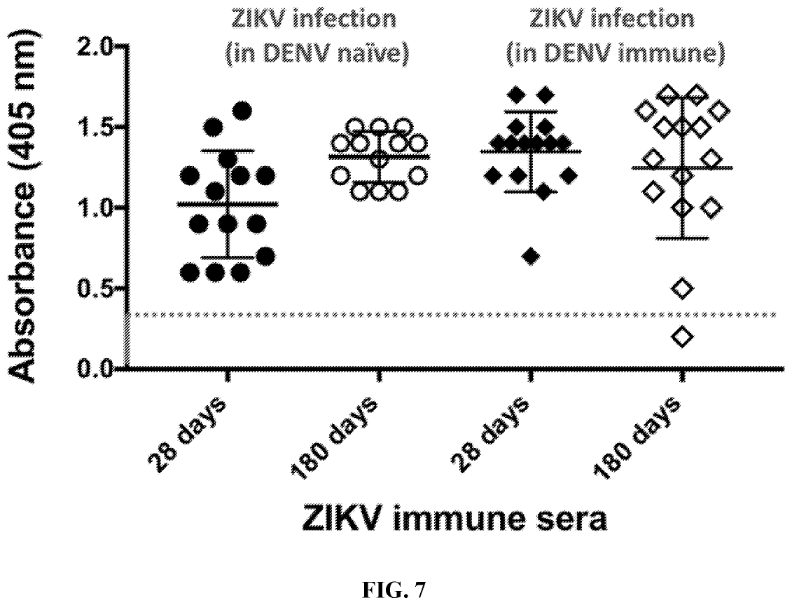

This example describes the sensitivity of detection and the kinetics of serum IgG antibodies in Zika-infected patients by Z-EDIII ELISA described in Example 1. To examine the kinetics of serum IgG antibodies that can be detected by Z-EDIII ELISA, we used 86 archived samples collected between 1- and 190-days post-infection from 25 PCR confirmed cases of primary and secondary Zika virus infection in the endemic area. Data presented in FIG. 6 demonstrated that Z-EDIII targeting serum IgG antibodies could be detected as early as first-week post infection in patients with either primary or secondary ZIKV infection. To examine the sensitivity of Z-EDIII ELISA for detection of acute and remote ZIKV infection in DENV naïve and DENV immune people, we analyzed sequential ZIKV immune serum samples collected around 28 days and 180 days post-infection from 29 DENV naïve and DENV immune patients by Z-EDIII ELISA. As demonstrated in FIG. 7 that Z-EDIII ELISA sensitivity remained over 95% during acute (29/29) and remote (28/29) ZIKV infection in DENV naïve and DENV immune patients.

### Example 3. Reducing Heterophilic Antibody Interference in Z-EDIII ELISA

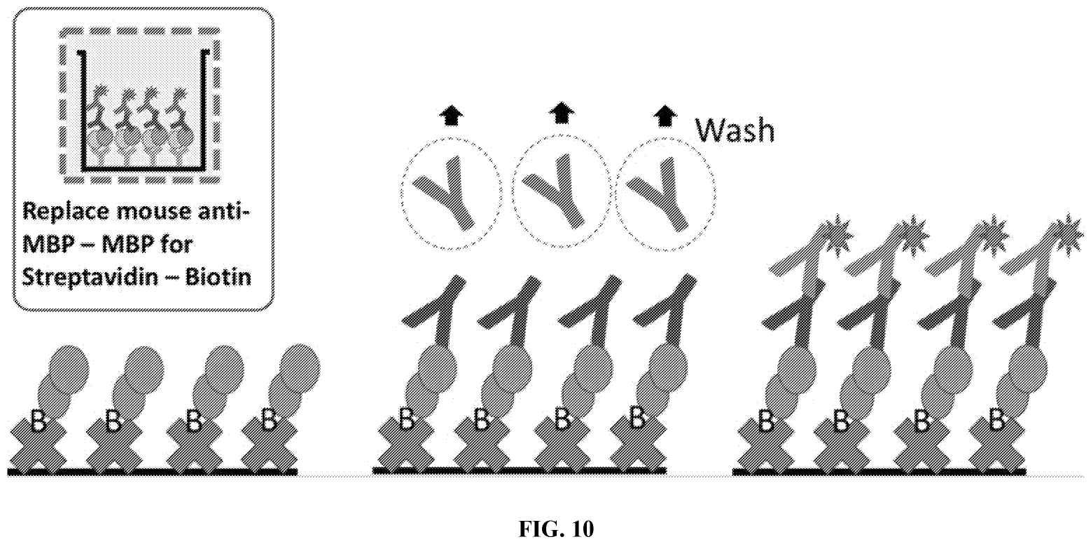

We demonstrate here that reformatting Z-EDIII assay based on “antibody-MBP-EDIII” capture (described in Example 1) with “streptavidin-biotin-EDIII conjugate” capture is an effective strategy for reducing assay interference. The Z-EDIII ELISA described in example 1 relied on a mouse anti-MBP capture antibody to immobilize Z-EDIII antigen on a plate. As with traditional sandwich ELISA diagnostics, we observed endogenous antibodies in some patient sera that caused interference with mouse anti-MBP capture antibody. Besides, this assay format did not permit analyzing mouse serum samples obtained for example from Zika virus vaccine efficacy studies as capture antibody used was of mouse origin (anti-MBP). Heterophilic antibodies in some patient sera can display weak multispecific binding directed against the Fc portion of immunoglobulin G (IgG) molecules used to capture antigen from other sources. Heterophilic antibodies can result in significant nonspecific background signal and false positives in sandwich ELISA. Several strategies have been used to reduce heterophilic antibodies interference in sandwich ELISAs including the use of blocking agents. An alternate approach to eliminate the heterophilic antibody interference, as well as to improve assay performance for detecting recent ZIKV infection by homologous antigen competition, in the Z-EDIII assay is shown in FIG. 10. As shown schematically, antibody-MBP-EDIII capture step is replaced with the capture based on a streptavidin-biotin-EDIII conjugate system to reduce heterophilic antibody interference.

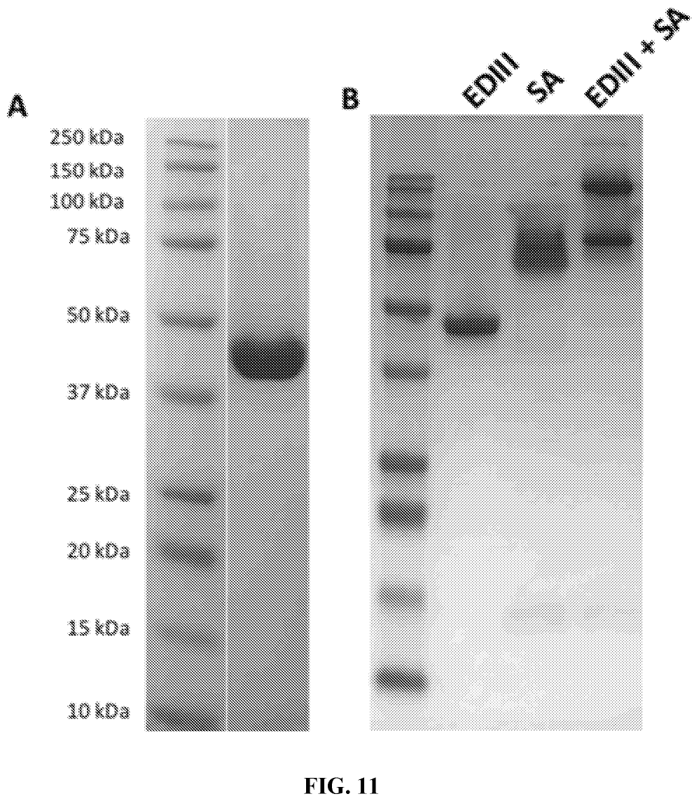

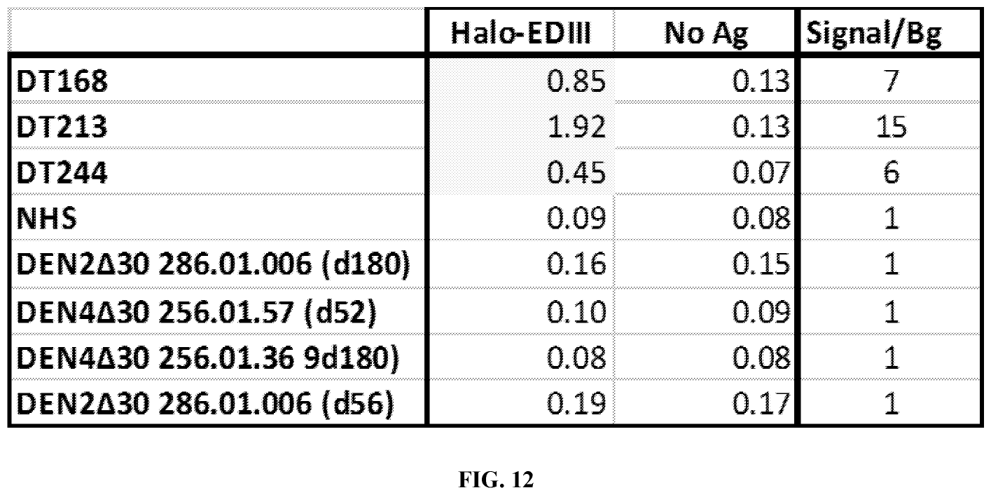

Next, we designed and produced Z-EDIII with an amino-terminal Halo-tag fusion in mammalian cells to facilitate site-specific labeling of Z-EDIII with Halo-tag biotin ligand, which comprises the HaloTag reactive linker and biotin (Promega). The HaloTag biotin ligand has been used to label and to capture a protein of interest fused to Halo-tag on solid supports using the strong biotin-streptavidin interaction (FIG. 11). As demonstrated by Coomassie-stained SDS-PAGE analysis, the Halo-tag fused Z-EDIII can be highly purified to homogeneity using metal affinity chromatography (FIG. 11, Panel A). Subsequent, gel-shift SDS-PAGE analysis, run under the non-reducing condition without boiling a sample of biotin-labeled Z-EDIII mixed with streptavidin, confirmed highly efficient, site-specific labeling of Z-EDIII with biotin (FIG. 11, Panel B). The evaluation of biotin-labeled Z-EDIII antigen with a collection of Zika immune serum and DENV immune serum, which previously gave background signal with the assay described in Example 1, is shown in FIG. 12. Taken together, the assay described in FIG. 10 can significantly reduce the heterophilic antibody interference in the Z-EDIII ELISA.

### Example 4. Development of NS1 Antigen Competition (NS1 cELISA) Assay to Serologically Differentiate Zika from Dengue Virus Infection

NS1 based antibody detection assay is an alternative and potentially attractive strategy for serological detection of ZIKV infection. However, ZIKV NS1 protein shares 54-57% sequence identity with NS1 proteins from different clinically relevant flaviviruses and contains conserved epitopes that can lead to extensive cross-reactivity with polyclonal serum antibodies. Accordingly, full-length NS1 based IgG ELISA has been reported with notable false-positivity in some settings. To overcome this drawback, we developed a Z-NS1 antigen competition ELISA assay (NS1 cELISA) for differential serological detection of recent and remote ZIKV infection, which is described here.

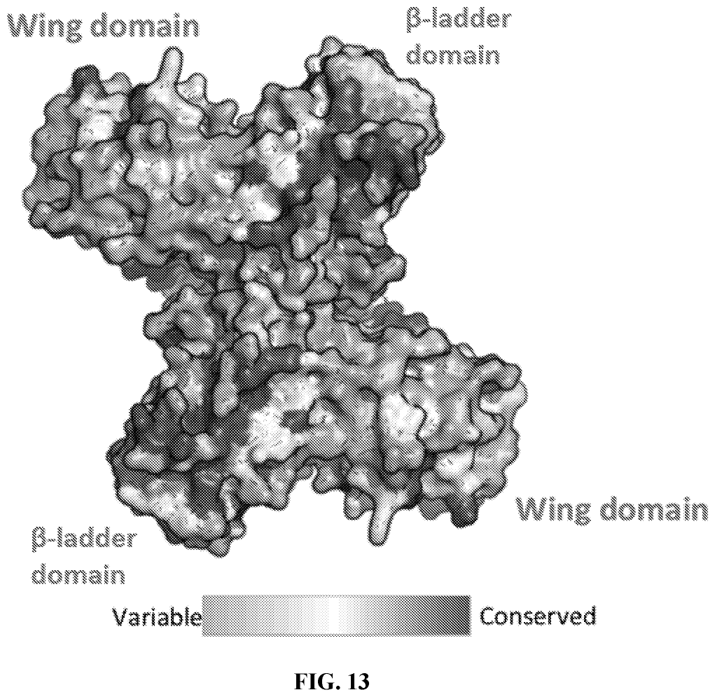

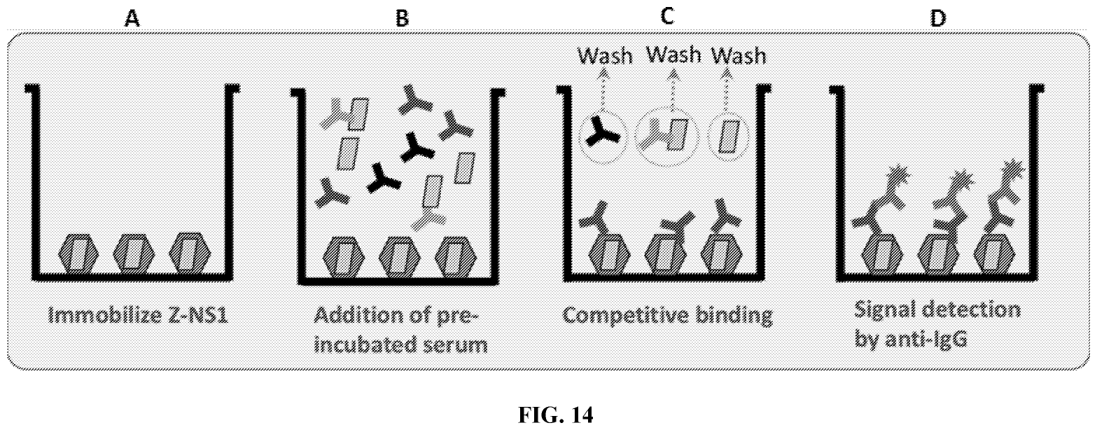

To identify NS1 protein antigenic regions that may be targeted for ZIKV-specific and flavivirus cross-reactive antibodies, we generated and compared a map of surface amino acid conservation between different flaviviruses including ZIKV, the 4 DENV serotypes, West Nile virus and Yellow fever virus using the Consurf algorithm (FIG. 13). Surface amino acid conservation analysis has been used as a tool to identify conserved and variable regions in protein. Our rationale is that combining surface conservation analysis with the knowledge of epitope information can provide valuable information to identify ZIKV-specific antigenic regions. Projecting the Consurf conservation score onto the molecular surface of the ZIKV NS1 showed (FIG. 13) that the C-terminal β-ladder domain is highly conserved whereas the wing domain is highly divergent between flaviviruses. Since previous studies have described many epitopes within the β-ladder domain, we hypothesized that the highly conserved C-terminal β-ladder domain contributes to serological cross-reactivity of NS1 antigen. Guided by our surface conversation analysis shown in FIG. 13, we developed a Z-NS1 antigen competition ELISA, which achieves specificity by pitting between plate-immobilized full-length Z-NS1 and Z-NS1 β-ladder domain in solution for binding to Z-NS1 specific serum antibody (FIG. 14). In this assay, a serum sample is first pre-incubated with recombinant Z-NS1 β-ladder domain (contains the high degree of conserved surface patches and cross-reactive epitopes) at high concentration to form serum antibodies targeting β-ladder domain to antigen-antibody complex. The pre-incubated serum with the β-ladder domain is then added to microtiter plates coated with full-length ZIKV NS1. The wells are washed to eliminate NS1 β-ladder domain-antibody complexes (which include a population of serum cross-reactive antibodies) and free Z-NS1 β-ladder domain. The bound serum IgG antibodies, most likely targeting the wing domain and the quaternary epitopes involving the wing domain, are detected using a secondary antibody conjugated to a detection reagent, which can be, for example, alkaline phosphatase. Since cross-reactive antibodies to NS1 are eliminated by Z-NS1 β-ladder domain antigen competition, the NS1 cELISA described here can be used for differential detection of recent and remote ZIKV infection.

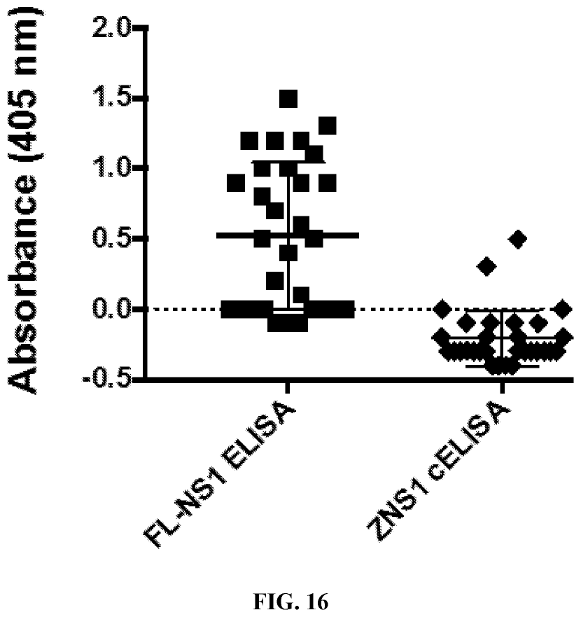

To evaluate Z-NS1 cELISA for differential serological detection of ZIKV infection, we assembled 30 characterized human secondary DENV immune sera collected between 2- and 9-weeks post-infection from patients living in the endemic areas. As demonstrated in FIG. 16, our Z-NS1 cELISA identified correctly 28 of 30 samples as true negative, yielding an assay specificity of over 93%. In comparison, specificity of the most commonly described full-length NS1 ELISA was less than 50%. Consequently, the Z-NS1 cELISA demonstrated here can be a powerful diagnostic tool to distinguish Zika virus infection from dengue virus and other flavivirus infection.

All publications, patent applications, patents, accession numbers and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

### Sequence Details of the Zika Antigens:

Zika EDI is fused to maltose binding protein (MBP)
for periplasmic expression in E. coli.
(SEQ ID NO: 1)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSNNNNNNNNNNLGIEENLYFQSNAIRCIGVSNRDFVEGMSGGT
WVDVVLEHGGCVTVMAQDKPTVDIELVTTTNGEYRIMLSVHGSQHSGMIV
NDTGHETDENRAKVEITPNSPRAEATLGGFGSLGLDCEPRTGSGHLKCRL
KMDKLRLKG
Zika EDIII is fused to maltose binding protein
(MBP) for periplasmic expression in E. coli.
(SEQ ID NO: 2)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNLGIEENLYFQSNAGVSYSLCTAAFTFTKIPA
ETLHGTVTVEVQYAGTDGPCKVPAQMAVDMQTLTPVGRLITANPVITEST
ENSKMMLELDPPFGDSYIVIGVGEKKITHHWHRS
Zika EDIII variant, which lacks two cross-reactive
epitopes (ABDE epitope and C-C′ epitope) but
fully retains the Zika specific epitopes.
(SEQ ID NO: 3)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNLGIEENLYFQSNAGVSYSLCTAAFTFTKHPA
ETGHGTVQVEVQYAGTDGPCKVPAQMATDLNDLTPVGRLITANPVITEST
ENSKMMLELDPPFGDSYIVIGVGEKKITHHWHRS
Zika EDII is fused to Halo-tag for mammalian
expression
(SEQ ID NO: 4)
MAEIGTGFPFDPHYVEVLGERMHYVDVGPRDGTPVLFLHGNPTSSYVWRN
IIPHVAPTHRCIAPDLIGMGKSDKPDLGYFFDDHVRFMDAFIEALGLEEV
VLVIHDWGSALGFHWAKRNPERVKGIAFMEFIRPIPTWDEWPEFARETFQ
AFRTTDVGRKLIIDQNVFIEGTLPMGVVRPLTEVEMDHYREPFLNPVDRE
PLWRFPNELPIAGEPANIVALVEEYMDWLHQSPVPKLLFWGTPGVLIPPA
EAARLAKSLPNCKAVDIGPGLNLLQEDNPDLIGSEIARWLSTLEISGGGG
GSGGGIEENLYFQSNAGVSYSLCTAAFTFTKIPAETLHGTVTVEVQYAGT
DGPCKVPAQMAVDMQTLTPVGRLITANPVITESTENSKMMLELDPPFGDS
YIVIGVGEKKITHHWHRSGSSGGSLPETGGHHHHHH
Zika NS1 Full-length with C-terminal His-tag for
expression in mammalian cells
(SEQ ID NO: 5)
DVGCSVDFSKKETRCGTGVFVYNDVEAWRDRYKYHPDSPRRLAAAVKQAW
EDGICGISSVSRMENIMWRSVEGELNAILEENGVQLTVVVGSVKNPMWRG
PQRLPVPVNELPHGWKAWGKSYFVRAAKTNNSFVVDGDTLKECPLKHRAW
NSFLVEDHGFGVFHTSVWLKVREDYSLECDPAVIGTAVKGKEAVHSDLGY
WIESEKNDTWRLKRAHLIEMKTCEWPKSHTLWTDGIEESDLIIPKSLAGP
LSHHNTREGYRTQMKGPWHSEELEIRFEECPGTKVHVEETCGTRGPSLRS
TTASGRVIEEWCCRECTMPPLSFRAKDGCWYGMEIRPRKEPESNLVRSMV
TAGGHHHHHH
Zika NS1 Full-length with N-terminal His-tag for
expression in mammalian cells
(SEQ ID NO: 6)
GHHHHHHDVGCSVDFSKKETRCGTGVFVYNDVEAWRDRYKYHPDSPRRLA
AAVKQAWEDGICSSVSRMNIMWRSVEGELNAILEENGVQLTVVVGSVKNP
MWRGPQRLPVPVNELPHGWKAWGKSYFVRAAKTNNSFVVDGDTLKECPLK
HRAWNSFLVEDHGFGVFHTSVWLKVREDYSLECDPAVIGTAVKGKEAVHS
DLGYWIESEKNDTWRLKRAHLIEMKTCEWPKSHTLWTDGIEESDLIIPKS
LAGPLSHHNTREGYRTQMKGPWHSEELEIRFEECPGTKVHVEETCGTRGP
SLRSTTASGRVIEEWCCRECTMPPLSFRAKDGCWYGMEIRPRKEPESNLV
RSMVTA
Zika C-terminal NS1 is fused to maltose binding
protein (MBP) for periplasmic expression in
E. coli
(SEQ ID NO: 7)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNLGIEENLYFQSNAREDYSLECDPAVIGTAVK
GKEAVHSDLGYWIESEKNDTWRLKRAHLIEMKTCEWPKSHTLWTDGIEES
DLIIPKSLAGPLSHHNTREGYRTQMKGPWHSEELEIRFEECPGTKVHVEE
TCGTRGPSLRSTTASGRVIEEWCCRECTMPPLSFRAKDGCWYGMEIRPRK
EPESNLVRSMVTA

TABLE 1
Characteristics of late convalescent serum
specimens collected >12 weeks post infection

| Serum | | Location of | Days post | |
|---|---|---|---|---|
| ID | Serum Category | Infection | infection | |
| DT168 | Primary ZIKV | Brazil | >6 | months |
| DT172 | Primary ZIKV | Colombia | 3 | months |
| DT206b | Primary ZIKV | Honduras | 6 | months |
| DT244b | Primary ZIKV | Puerto Rico | 6 | months |
| DT166 | Secondary DENV + ZIKV | Brazil | >6 | months |
| ARB15 | Secondary DENV + ZIKV | Brazil | 6 | months |
| ARB24 | Secondary DENV + ZIKV | Brazil | 6 | months |
| ARB14 | Secondary DENV + ZIKV | Brazil | 6 | months |
| ARB19 | Secondary DENV + ZIKV | Brazil | 8 | months |
| ARB17 | Secondary DENV + ZIKV | Brazil | 5 | months |
| DT147 | Primary DENV1 | Bolivia | 2 | years |
| DT153 | Primary DENV1 | Guyana | 5 | years |
| DT118 | Primary DENV3 | Nicaragua | 1 | year |
| DT125 | Primary DENV3 | Paraguay | 5 | years |
| ARB27 | Primary DENV2 | Brazil | 8 | months |
| DT033 | Primary DENV3 | India | >4 | months |
| ARB16 | Secondary DENV | Brazil | 7 | months |
| ARB22 | Secondary DENV | Brazil | 6 | months |
| DT026 | Secondary DENV | Dominican Republic | 1.5 | years |
| DT146 | Secondary DENV | India | 1 | year |
| DT141 | Secondary DENV | Dominican Republic | 8 | years |
| DT160 | Secondary DENV | Puerto Rico | 1 | year |
| DT140 | Naïve human sera | | | |
| DT127 | Naïve human sera | | | |

TABLE 2
Characteristics of early convalescent serum specimens
collected between 2 and 12 weeks post infection

| Serum | | Location of | Days post |
|---|---|---|---|
| ID | Serum Category | Infection | infection |
| 5604.12.B.2 | Primary ZIKV | Nicaragua | 14 |
| 5847.12.B.2 | Primary ZIKV | Nicaragua | 15 |
| 5749.12.A.2 | Primary ZIKV | Nicaragua | 20 |
| 8385.12.B.2 | Primary ZIKV | Nicaragua | 23 |
| 7420.12.B.2 | Secondary ZIKV | Nicaragua | 14 |
| LB17 | Secondary ZIKV | Colombia | 70 |
| LB18 | Secondary ZIKV | Colombia | 70 |
| LB19 | Secondary ZIKV | Colombia | 70 |
| LB20 | Secondary ZIKV | Colombia | 70 |
| LB22 | Secondary ZIKV | Colombia | 70 |
| LB23 | Secondary ZIKV | Colombia | 70 |
| GS0437-2 | Primary DENV | Sri Lanka | 11 |
| GS0505-2 | Primary DENV | Sri Lanka | 19 |
| GS0409-2 | Primary DENV | Sri Lanka | 24 |
| GS0517-2 | Primary DENV | Sri Lanka | 30 |
| GS0518-2 | Primary DENV | Sri Lanka | 30 |
| GS0523-2 | Primary DENV | Sri Lanka | 44 |
| GS0399-2 | Primary DENV | Sri Lanka | 60 |
| GS0402-2 | Secondary DENV | Sri Lanka | 12 |
| GS0438-2 | Secondary DENV | Sri Lanka | 18 |
| GS0504-2 | Secondary DENV | Sri Lanka | 23 |
| GS0519-2 | Secondary DENV | Sri Lanka | 23 |
| GS0509-2 | Secondary DENV | Sri Lanka | 34 |
| GS0521-2 | Secondary DENV | Sri Lanka | 77 |

TABLE 3

| | Abbreviation | |
|---|---|---|
| | Three-Letter | One-Letter Code (can be |
| Amino Acid Residue | Code | upper or lower case) |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 4

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

1. A recombinant polypeptide for use in an immunoassay, comprising an amino acid sequence selected from the group consisting of:
a)
(SEQ ID NO: 1)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNNLGIEENLYFQSNAIRCIGVSNRDFVEGMSG
GTWVDVVLEHGGCVTVMAQDKPTVDIELVTTTNGEYRIMLSVHGSQHSGM
IVNDTGHETDENRAKVEITPNSPRAEATLGGFGSLGLDCEPRTGSGHLKC
RLKMDKLRLKG (EDI-MBP);
b)
(SEQ ID NO: 2)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNNLGIEENLYFQSNAGVSYSLCTAAFTFTKIP
AETLHGTVTVEVQYAGTDGPCKVPAQMAVDMQTLTPVGRLITANPVITES
TENSKMMLELDPPFGDSYIVIGVGEKKITHHWHRS (EDIII-MBP);
c)
(SEQ ID NO : 3)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNNLGIEENLYFQSNAGVSYSLCTAAFTFTKHP
AETGHGTVQVEVQYAGTDGPCKVPAQMATDLNDLTPVGRLITANPVITES
TENSKMMLELDPPFGDSYIVIGVGEKKITHHWHRS (EDIII
variant-MBP);
d)
(SEQ ID NO: 4)
MAEIGTGFPFDPHYVEVLGERMHYVDVGPRDGTPVLFLHGNPTSSYVWRN
IIPHVAPTHRCIAPDLIGMGKSDKPDLGYFFDDHVRFMDAFIEALGLEEV
VLVIHDWGSALGFHWAKRNPERVKGIAFMEFIRPIPTWDEWPEFARETFQ
AFRTTDVGRKLIIDQNVFIEGTLPMGVVRPLTEVEMDHYREPFLNPVDRE
PLWRFPNELPIAGEPANIVALVEEYMDWLHQSPVPKLLFWGTPGVLIPPA
EAARLAKSLPNCKAVDIGPGLNLLQEDNPDLIGSEIARWLSTLEISGGGG
GSGGGIEENLYFQSNAGVSYSLCTAAFTFTKIPAETLHGTVTVEVQYAGT
DGPCKVPAQMAVDMQTLTPVGRLITANPVITESTENSKMMLELDPPFGDS
YIVIGVGEKKITHHWHRSGSSGGSLPETGGHEIHHHH (EDIII-Halo
tag);
e)
(SEQ ID NO: 5)
DVGCSVDFSKKETRCGTGVFVYNDVEAWRDRYKYHPDSPRRLAAAVKQAW
EDGICGISSVSRMENIMWRSVEGELNAILEENGVQLTVVVGSVKNPMWRG
PQRLPVPVNELPHGWKAWGKSYFVRAAKTNNSFVVDGDTLKECPLKHRAW
NSFLVEDHGFGVFHTSVWLKVREDYSLECDPAVIGTAVKGKEAVHSDLGY
WIESEKNDTWRLKRAHLIEMKTCEWPKSHTLWTDGIEESDLIIPKSLAGP
LSHHNTREGYRTQMKGPWHSEELEIRFEECPGTKVHVEETCGTRGPSLRS
TTASGRVIEEWCCRECTMPPLSFRAKDGCWYGMEIRPRKEPESNLVRSMV
TAGGHHHHHH (NS1-C terminal His tag);
f)
(SEQ ID NO: 6)
GHHHHHHDVGCSVDFSKKETRCGTGVFVYNDVEAWRDRYKYHPDSPRRLA
AAVKQAWEDGICSSVSRMNIMWRSVEGELNAILEENGVQLTVVVGSVKNP
MWRGPQRLPVPVNELPHGWKAWGKSYFVRAAKTNNSFVVDGDTLKECPLK
HRAWNSFLVEDHGFGVFHTSVWLKVREDYSLECDPAVIGTAVKGKEAVHS
DLGYWIESEKNDTWRLKRAHLIEMKTCEWPKSHTLWTDGIEESDLIIPKS
LAGPLSHHNTREGYRTQMKGPWHSEELEIRFEECPGTKVHVEETCGTRGP
SLRSTTASGRVIEEWCCRECTMPPLSFRAKDGCWYGMEIRPRKEPESNLV
RSMVTA (NS1-N terminal His tag);
g)
(SEQ ID NO: 7)
MKIKTGARILALSALTTMMFSASALAKSSHHHHHHGSSMKIEEGKLVIWI
NGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVE
ALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYS
IAEAAFNKGETAMTINGPWAWSNIDTSKVNYGVTVLPTFKGQPSKPFVGV
LSAGINAASPNKELAKEFLENYLLTDEGLEAVNKDKPLGAVALKSYEEEL
AKDPRIAATMENAQKGEIMPNIPQMSAFWYAVRTAVINAASGRQTVDEAL
KDAQTNSSSNNNNNNNNNNGIEENLYFQSNAREDYSLECDPAVIGTAVKG
KEAVHSDLGYWIESEKNDTWRLKRAHLIEMKTCEWPKSHTLWTDGIEESD
LIIPKSLAGPLSHHNTREGYRTQMKGPWHSEELEIRFEECPGTKVHVEET
CGTRGPSLRSTTASGRVIEEWCCRECTMPPLSFRAKDGCWYGMEIRPRKE
PESNLVRSMVTA (C-terminal NS1-MBP);
and
h)
any combination of (a)-(g).

2. A nucleic acid molecule encoding the recombinant polypeptide of claim 1.

3. A composition comprising the recombinant polypeptide of claim 1 in a pharmaceutically acceptable carrier.

4. The recombinant polypeptide of claim 1 bound to a solid support.

5. The recombinant polypeptide of claim 1 linked to a detectable moiety.

6. A method of diagnosing a Zika virus infection in a subject, comprising:
a) contacting a sample from the subject with the recombinant polypeptide of claim 1 under conditions whereby an antigen/antibody complex can form; and
b) detecting formation of the antigen/antibody complex, thereby diagnosing a Zika virus infection in the subject.

7. A method of detecting an antibody to Zika virus in a sample, comprising:
a) contacting the sample with the recombinant polypeptide of claim 1 under conditions whereby an antigen/antibody complex can form; and
b) detecting formation of the antigen/antibody complex, thereby detecting an antibody to Zika virus in the sample.

8. A method of identifying an infection by Zika virus in a subject known to have, or suspected of having, a flavivirus infection, comprising:
a) contacting a sample from the subject with the recombinant polypeptide of claim 1 under conditions whereby an antigen/antibody complex can form; and
b) detecting formation of the antigen/antibody complex, thereby identifying an infection by Zika virus in the subject.

9. The method of claim 6, wherein the method is carried out in an immunoassay.

10. The method of claim 9, wherein the immunoassay is an enzyme linked immunosorbent assay (ELISA).

11. The method of claim 9, wherein the immunoassay is a lateral flow assay (LFA).

12. The method of claim 9, wherein the immunoassay is a multiplex assay.

13. The method of claim 12, wherein the multiplex assay is a plasmonic gold platform.

14. The method of claim 12, wherein the multiplex assay is a microbead-based assay.

15. The method of claim 9, wherein the immunoassay is a competitive binding assay.

16. A method of identifying an infection by Zika virus in a subject, comprising:
a) contacting a serum sample from the subject with a recombinant Z-NS1 β-ladder domain under conditions whereby an antigen/antibody complex can form;
b) contacting the serum sample comprising the recombinant Z-NS1 β-ladder domain of step (a) with a full length Zika NS1 polypeptide that is bound to a solid substrate in a reaction well under conditions whereby an antigen/antibody complex can form;
c) washing the reaction well of (b) to remove unbound antibody, unbound antigen/antibody complexes, and unbound recombinant Zika NS1 β-ladder domain; and
d) detecting the formation of an antigen/antibody complex comprising the full length Zika NS1 polypeptide bound to the solid substrate, thereby identifying an infection by Zika virus in the subject.

17. The method of claim 16, wherein the subject is known to have or is suspected of having a flavivirus infection.

18. The method of claim 7, wherein the method is carried out in an immunoassay.

19. The method of claim 8, wherein the method is carried out in an immunoassay.
